(19) United States
(12) Patent Application Publication
(10) Pub. No.: US 2011/0245265 A1
(43) Pub. Date: Oct. 6, 2011

(54) CXCR4 ANTAGONISTS FOR KIDNEY INJURY

(75) Inventors: Anna Zuk, Natick, MA (US); Steven Ledbetter, Westborough, MA (US); Nibedita Chattopadhyay, Ashland, MA (US)

(73) Assignee: GENZYME CORPORATION, Cambridge, MA (US)

(21) Appl. No.: 13/061,087

(22) PCT Filed: Aug. 28, 2009
(86) PCT No.: PCT/US2009/055437

§ 371 Date: May 24, 2011

Related U.S. Application Data
(60) Provisional application No. 61/093,273, filed on Aug. 29, 2008.
(60) Provisional application No. 61/112,637, filed on Nov. 7, 2008.
(60) Provisional application No. 61/187,612, filed on Jun. 16, 2009.

(51) Int. Cl. A61K 31/496; A61K 31/4427; A61K 31/395; A61K 31/444; A61K 31/497; A61K 31/4178; A61K 31/52; A61P 13/12

(52) U.S. Cl. 514/252.13; 514/340; 514/183; 514/444; 514/333; 514/255.05; 514/397; 514/263.2

(57) ABSTRACT

Methods to treat or prevent acute kidney injury and chronic kidney injury in subjects using CXCR4 antagonists are disclosed.

### TECHNICAL FIELD

The invention is in the field of therapeutics and medicinal chemistry. In particular, the invention concerns methods of treatment and prevention of chronic and acute kidney injury by administering certain polyamines.

### BACKGROUND ART

Acute Kidney Injury (AKI) is the sudden deterioration of kidney function. In particular, AKI results in the failure of kidneys to excrete nitrogenous waste, such as urea and creatine, failure to maintain fluid and electrolyte balance as well as loss of ability to concentrate urine. Among the symptoms of AKI are an increase in serum creatine and a decrease in urine output. Other metabolic disturbances include metabolic acidosis and hyperkalemia.

The cause of AKI is related in an estimated 60% of cases to ischemia from surgery, vascular disease, trauma, burns or sepsis, for example. In another 40% of cases, AKI results from toxins, such as from radiocontrast dyes, NSAIDs, antimicrobials, antifungals, cyclosporine, aminoglycosides, and chemotherapy. Other possible causes of kidney failure include disruption of blood flow to the kidneys from hypovolemia; dehydration from vomiting, diarrhea, water pills or blood loss; vascular problems such as heart failure or heart attacks; or liver failure, for example. Kidney failure may also be caused by obstructions in the kidney caused from kidney/bladder stones; cancer of the urinary tract or related structures; or blood clots. In addition, AKI may occur from primary renal damage wherein the filtering function of the kidney, the blood supply within the kidney or the processing ability of the kidney tissue is affected.

In some cases AKI is the result of an ischemia-reperfusion injury in which the sequence of cold and warm ischemia and subsequent reperfusion of donor kidneys prior to transplantation leads to delayed graft function in the recipient. AKI can also occur when a kidney is transplanted and has not yet begun to function in the organ recipient, a condition called “delayed graft function.” Recipients of a transplant that exhibits delayed graft function are at increased risk of developing chronic allograft nephropathy and graft failure. This particular type of injury is one of contributing factors to chronic transplant rejection and why greater than 50% of transplants that survive the 1 year mark fail after 5-10 years transplant rejection.

Discussion of therapies for the treatment of ischemia-reperfusion injuries can be found in the following resources: Myocardial Infarct: Misao, Y, G. Takemura, M. Arai, et al. 2006 “Importance of recruitment of bone marrow-derived CXCR4+ cells in post-infarct cardiac repair mediated by G-CSF” Cardiovasc. Res. 71: 455-465; Peripheral Limb Ischemia: Jiao, C., S. Fricker, and G. C. Schatteman. 2006. “The chemokine (CXC motif) receptor 4 inhibitor AMD3100 accelerates blood flow restoration in diabetic mice” Diabetologia 49:2786-2789. Capoccia, B J., R. M. Shepherd, and D. C. Link. 2006 “G-CSF and AMD3100 mobilize monocytes into the blood that stimulate angiogenesis in vivo through a paracrine mechanism” Blood 108: 2438-2445; Discussion of therapies for treatment of inflammation can be found in the following resources: Rheumatoid Arthritis Models: DeClercq, B., L. Geboes, S. Hatse, et al. 2005 “Pro-inflammatory properties of stromal cell-derived factor-1 (CXCL12) in collagen-induced arthritis” Arthritis Research & Therapy 7:R1208-R1220; Matthys, P., S. Hatse, K. Vermeire, et al. 2001 “AMD3100, a potent and specific antagonist of the stromal cell-derived factor-1 chemokine receptor CXCR4, inhibits autoimmune joint inflammation in IFN-gamma receptor-deficient mice” J. Immunol. 167:4686-4692. Discussion of stem cells contributing to renal recovery can be found in Togel, F, J. Isaac, Z. Hu, et al. 2005 “Renal SDF-1 signals mobilization and homing of CXCR-4 positive cells to the kidney after ischemic injury” Kid. Int. 67: 1772-1784; Lange, C., F. Togel, H. Ittrich, et al. 2005 “Administered mesenchymal stem cells enhance recovery from ischemia/reperfusion-induced acute renal failure in rat” Kid. Int. 68: 1613-1617; Morigi, M., B. Imberti, C. Zoj a, et al. 2004 “Mesenchymal stem cells are renotropic, helping to repair the kidney and improve function in acute renal failure” J. Am. Soc. Nephrol. 15: 1794-1804; Dekel, B., E. Shezen, S. Even-Toy-Friedman, et al. 2006 “Transplantation of human hematopoietic stem cells into ischemic and growing kidneys suggests a role in vaculogenesis but not tubulogenesis” Stem Cells 24: 1185-1193; B. Bi, R. Schmitt, M. Israilova, et al. 2007 “Stromal cells protect against acute tubular injury via an endocrine effect” J. Am. Soc. Nephrol. 18:2486-2496. Discussion of possible mechanisms of glomerulonephritis include: Ding, M., et al. 2006 “Loss of the tumor suppressor Vhlh leads to upregulation of CXCR4 and rapidly progressive glomerulonephritis in mice” Nature Med. 12, 9:1081-1087.

A high mortality rate, estimated at 40-50%, exists among patients afflicted with AKI. The mortality rate is even higher in patients in the ICU requiring hemodialysis and is roughly greater than 60%. The prognosis of the survivors depends on the existing renal status of the patient. The mortality rates for afflicted AKI individuals have been unchanged for nearly 40 years.

AKI can also lead to end stage renal disease (ESRD) and chronic kidney disease (CKD). Ishani, A., et al., Acute Kidney Injury Increases Risk of ESRD among Elderly., J. Am. Soc. Nephrol (2009). Okusa, Mark D., et al., The Nexus of Acute Kidney Injury, Chronic Kidney Disease, and World Kidney Day 2009., Clin. J. Am. Soc. Nephrol. (4) 520-522, 2009.

The incidence of AKI in 2002 was estimated to be about 600,000 patients, and 2% relating to hospital admissions. The reported incidence of AKI represents a 20-fold increase in 20 years. At $10 billion per year in related costs, AKI is a costly condition with no current pharmacological treatment available. AKI is an unmet medical need.

### DISCLOSURE OF THE INVENTION

In one aspect, the invention is directed to methods of treating animal subjects, in particular, veterinary and human subjects, that have or will develop an acute kidney injury (AKI) and chronic kidney injury. Thus, the methods include both prevention and treatment of AKI. The methods of the invention employ CXCR4 antagonists which, in one embodiment, are polyamines including those described in the patents and publications incorporated hereinbelow by reference.

In one aspect, the invention is directed to a method for preventing or treating acute kidney injury (AKI) in a subject which method comprises administering to said subject an antagonist of CXCR4. In certain embodiments, the antagonist is a compound of the formula

Z-linker-Z′ (1)

or a pharmaceutically acceptable salt or prodrug form thereof

wherein Z is a cyclic polyamine containing 9-32 ring members of which 2-8 are nitrogen atoms, said nitrogen atoms separated from each other by at least 2 carbon atoms, and wherein said heterocycle may optionally contain additional heteroatoms besides nitrogen and/or may be fused to an additional ring system;

or Z is of the formula

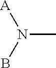

wherein A comprises a monocyclic or bicyclic fused ring system containing at least one N and B is H or an organic moiety of 1-20 atoms,

Z′ may be embodied in a form as defined by Z above, or alternatively may be of the formula

—N(R)—(CR₂)ₙ—X

wherein each R is independently H or straight, branched or cyclic alkyl (1-6C), n is 1 or 2, and X is an aromatic ring, including heteroaromatic rings, or is a mercaptan or Z′ may be

—Ar(Y)ⱼ;

wherein Ar is an aromatic or heteroaromatic moiety, and each Y is independently a non-interfering substituent and j is 0-3; and

“linker” represents a bond, alkylene (1-6C) or may comprise aryl, fused aryl, oxygen atoms contained in an alkylene chain, or may contain keto groups or nitrogen or sulfur atoms;

in an amount effective to prevent or treat acute kidney injury in said subject.

In another aspect, the invention is directed to a method for preventing or treating chronic kidney injury (CM) in a subject which method comprises administering to said subject an antagonist of CXCR4. In certain embodiments, the antagonist is a compound of the formula

Z-linker-Z′ (1)

or a pharmaceutically acceptable salt or prodrug form thereof

wherein Z is a cyclic polyamine containing 9-32 ring members of which 2-8 are nitrogen atoms, said nitrogen atoms separated from each other by at least 2 carbon atoms, and wherein said heterocycle may optionally contain additional heteroatoms besides nitrogen and/or may be fused to an additional ring system;

or Z is of the formula

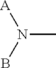

wherein A comprises a monocyclic or bicyclic fused ring system containing at least one N and B is H or an organic moiety of 1-20 atoms,

Z′ may be embodied in a form as defined by Z above, or alternatively may be of the formula

—N(R)—(CR₂)ₙ—X

wherein each R is independently H or straight, branched or cyclic alkyl (1-6C), n is 1 or 2, and X is an aromatic ring, including heteroaromatic rings, or is a mercaptan or Z′ may be

—Ar(Y)ⱼ;

wherein Ar is an aromatic or heteroaromatic moiety, and each Y is independently a non-interfering substituent and j is 0-3; and

“linker” represents a bond, alkylene (1-6C) or may comprise aryl, fused aryl, oxygen atoms contained in an alkylene chain, or may contain keto groups or nitrogen or sulfur atoms;

in an amount effective to prevent or treat acute kidney injury in said subject.

Irrespective of whether kidney injury (acute or chronic) is being treated or prevented, suitable CXCR4 antagonists include Mozobil(plerixafor) (f/k/a AMD3100); AMD3465; BKT140, including those CXCR4 antagonists described in U.S. Pat. No. 7,423,007 and U.S. Pub. No. 20040171552A1; KRH-2731/CS-3955, including those CXCR4 antagonists described in WO-2006095542 and WO/02094261; AVR 118; TG-0054, including those CXCR4 antagonists described in U.S. Pub. No. 20060160860A1, U.S. Pub. No. 20080058382, and U.S. Pat. No. 7,399,776; CTCE-0214 and CTCE-9908, including those CXCR4 antagonists described in WO 01/76615, WO 01/85196 and U.S. Ser. No. 11/649,928 and related applications; MSX-122; and POL-6326/POL-2438/POL-3026, including those CXCR4 antagonists described in WO 2008/104090, the contents of all the foregoing documents are hereby incorporated herein by reference for all purposes. In certain embodiments, the antagonist may be an antibody, such as a monoclonal antibody, or immunoreactive fragment thereof.

In another aspect, the invention is directed to a method for reducing the risk of an allograft rejection in an allograft transplant recipient who receives an allograft from an allograft donor. In certain embodiments, the donor is administered an antagonist of CXCR4. In certain embodiments, the recipient is administered an antagonist of CXCR4. In certain embodiments, the CXCR4 antagonist is administered to the organ preservation fluid ex vivo. Exemplary preservation fluids include but are not limited to University of Wisconsin (Belzer's) solution, Marshall's preservation fluid, Euro-Collins solution, and blood-based preservation fluids. Other types of organ preservation fluids will be readily apparent to those of skill in the art given the benefit of the present disclosure. In these embodiments, the method is accomplished by administering the CXCR4 antagonist to the allograft donor, allograft recipient and/or organ preservation fluid, as the case may be. As such, in accordance with this aspect, the CXCR4 antagonist can be administered to each of the donor, recipient or organ preservation fluid or any combination thereof. In certain embodiments, the CXCR4 antagonist is a compound of the formula

Z-linker-Z′ (1)

or a pharmaceutically acceptable salt or prodrug form thereof

wherein Z is a cyclic polyamine containing 9-32 ring members of which 2-8 are nitrogen atoms, said nitrogen atoms separated from each other by at least 2 carbon atoms, and wherein said heterocycle may optionally contain additional heteroatoms besides nitrogen and/or may be fused to an additional ring system;

or Z is of the formula

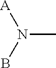

wherein A comprises a monocyclic or bicyclic fused ring system containing at least one N and B is H or an organic moiety of 1-20 atoms,

Z′ may be embodied in a form as defined by Z above, or alternatively may be of the formula

—N(R)—(CR₂)ₙ—X

wherein each R is independently H or straight, branched or cyclic alkyl (1-6C), n is 1 or 2, and X is an aromatic ring, including heteroaromatic rings, or is a mercaptan or Z′ may be

—Ar(Y)ⱼ;

wherein Ar is an aromatic or heteroaromatic moiety, and each Y is independently a non-interfering substituent and j is 0-3; and

“linker” represents a bond, alkylene (1-6C) or may comprise aryl, fused aryl, oxygen atoms contained in an alkylene chain, or may contain keto groups or nitrogen or sulfur atoms;

in an amount effective to reduce the risk of an allograft rejection in said recipient.

In another aspect, the invention is directed to methods for treating or preventing ESRD in a subject which method comprises administering to said subject an antagonist of CXCR4.

In another aspect, the invention is directed to methods for treating or preventing CKD in a subject which method comprises administering to said subject an antagonist of CXCR4.

In another aspect, the invention is directed to a method of treating or preventing kidney disease or onset of kidney disease in a subject afflicted with or at risk of developing kidney disease comprising administering to the subject an effective amount of an agonist of a down-regulated KD Marker or KD Polynucleotide (e.g. pVHL). The markers of kidney disease include one or more hypoxia related polypeptides, including von Hippel Lindau protein (pVHL), vascular endothelial growth factor A (VEGF-A), chemokine receptor chemokine (C—X—C motif) receptor 4 (CXCR4), hypoxia inducible factor alpha (HIF-1α), integrin-β-1, platelet-derived growth factor-A (PDGF-A), transforming growth factor beta (TGF β), and Interacting Polypeptides thereof. These markers including but not limited to native-sequence polypeptides, isoforms, chimeric polypeptides, all homologs, fragments, and precursors of the markers, and modified forms of the polypeptides and derivatives are referred to herein as “Kidney Disease Marker(s)” or “KD Markers”. Polynucleotides encoding KD Markers or expressing KD Markers are referred to herein as “Kidney Disease Polynucleotide Marker(s)”, “polynucleotides encoding kidney disease marker(s)” or “KD Polynucleotides”. The KD Markers and KD Polynucleotides are sometimes collectively referred to herein as “marker(s)”. Biomarkers have been identified for diagnosis and monitoring (i.e., monitoring progression or therapeutic treatment) of kidney diseases, in particular Rapid Progressive Glomerulonephritis (RPGN), more particularly pauci-immune RPGN.

In another aspect, the invention is directed to a method of treating or preventing kidney disease or onset of kidney disease in a subject having or at risk of developing kidney disease comprising administering to the subject an effective amount of an antagonist of an up-regulated KD Marker or KD Polynucleotide (e.g. CXCR4).

In another aspect, the invention is directed to a method of treating a subject afflicted with or at risk of developing kidney disease comprising inhibiting expression of one or more KD Marker or KD Polynucleotide, in particular CXCR4. In certain embodiments, the kidney disease being treated with the CXCR4 antagonist is RPGN or pauci-immune RPGN. Methods to determine whether a subject is afflicted with or at risk of developing any of the kidney diseases described herein such as RPGN or pauci-immune RPGN are readily available to a person of ordinary skill in the art given the benefit of this disclosure. In this regard, the present invention is directed to a method of treating kidney disease in a subject comprising administering to a subject in need thereof an antagonist of CXCR4. In certain embodiments, the kidney disease is RPGN. In certain embodiments, the kidney disease is pauci-immune RPGN. In certain embodiments, the CXCR4 antagonist is plerixafor.

In another aspect, the invention is directed to antibodies specific for KD Markers associated with kidney disease that can be used to inhibit KD Marker or KD Polynucleotide expression.

In another aspect, the invention is directed to methods for treating or preventing kidney disease or onset of kidney disease in a subject is provided comprising administering to a subject in need thereof antibodies specific for one or more up-regulated KD Markers, e.g., antibodies and small molecules that antagonize CXCR4 such as AMD3100.

In another aspect, the invention is directed to a method of using KD Markers or parts thereof, antibodies specific for KD Markers, or inhibitor of KD Polynucleotides (e.g. antisense) in the preparation or manufacture of a medicament for the prevention or treatment of kidney disease or onset of kidney disease.

The invention also provides a method for stimulating or enhancing in a subject production of antibodies directed against one or more up-regulated KD Marker (e.g. CXCR4). The method comprises administering to the subject one or more up-regulated KD Marker, peptides derived therefrom, or chemically produced (synthetic) peptides, or any combination of these molecules of the invention in a dose effective for stimulating or enhancing production of the antibodies.

The invention contemplates the methods, compositions, and kits described herein using additional markers associated with kidney disease. The methods described herein may be modified by including reagents to detect the additional markers, or polynucleotides for the markers.

Other features and advantages of the present inventions will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art given the benefit of this disclosure.

### BRIEF DESCRIPTION OF THE DRAWINGS

All of the data shown in the figures and throughout the specification represent mean+/− standard deviation. Any asterisk (*) shown in the accompanying figures indicates statistical significance, however, the lack of an asterisk (*) in the accompanying figures should not be taken to mean that there was no statistical significance.

FIG. 1A shows an overview of the dosing using AMD3465 and experimental timeline. FIGS. 1B and 1C show graphs of serum creatine levels and blood urea nitrogen (BUN) levels, respectively, of subjects treated with AMD3465. FIGS. 1D and 1E show the anti-inflammatory effects of this compound.

FIGS. 2A and 2B show images of tissue of subjects treated with and without AMD3465. FIGS. 2C and 2D show graphs of injury score and TUNEL score data, respectively, of subjects treated with AMD3465.

FIGS. 3A and 3B show tissue of subjects treated with and without AMD3465. FIGS. 3C and 3D show graphs of change in Rat IgG count and reduction of levels of secreted von Willebrand factor (vWF) in response to administration of AMD3465.

FIG. 4A shows a graph of body weight changes and FIG. 4B shows a graph of white blood cell (WBC) mobilization at 24 hours in subjects treated with AMD3465.

FIG. 5A shows an overview of the dosing and experimental timeline. FIGS. 5B and 5C show graphs of plasma creatinine levels and FIG. 5D shows blood urea nitrogen (BUN) levels of subjects treated with AMD3100.

FIG. 6A shows an overview of an alternative dosing and experimental timeline. FIGS. 6B and 6C show graphs of serum creatine levels and blood urea nitrogen (BUN) levels, respectively, of subjects treated with lower doses of AMD3100.

FIGS. 7A-7C show the protocol and results of a dosing regimen with lower dosage than FIGS. 6A-6C.

FIGS. 8A-8D show the results of the protocol of FIG. 6A on epithelial injury and death.

FIG. 9 shows the effect of AMD3100 on WBC mobilization.

### MODES OF CARRYING OUT THE INVENTION

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art. As appropriate, procedures involving the use of commercially available kits and reagents are generally carried out in accordance with manufacturer defined protocols and/or parameters unless otherwise noted.

The discussion of the general methods given herein is intended for illustrative purposes only. Other alternative methods and embodiments will be apparent to those of skill in the art upon review of this disclosure.

A group of items linked with the conjunction “or” should not be read as requiring mutual exclusivity among that group, but rather should also be read as “and/or” unless expressly stated otherwise. Although items, elements, or components of the invention may be described or claimed in the singular, the plural is contemplated to be within the scope thereof unless limitation to the singular is explicitly stated.

Acute kidney injury (AKI) in subjects may be determined by methods and criteria known in the art. For example, renal failure is diagnosed when either creatinine or blood urea nitrogen tests are elevated, especially when oliguria is present. Previous measurements of renal function may offer comparison, which is especially important if a patient is known to have chronic renal failure as well. Blood tests and examination of a urine specimen is typically performed to elucidate the cause of acute renal failure and medical ultrasonography of the renal tract may also be performed.

In some embodiments, subjects are at risk of developing or have developed acute kidney injury because of any of the following conditions: hypovolemia (decreased blood volume), usually from shock or dehydration and fluid loss or excessive diuretics use; hepatorenal syndrome in which renal perfusion is compromised in liver failure; vascular problems, such as atheroembolic disease and renal vein thrombosis (which can occur as a complication of the nephrotic syndrome); infection usually sepsis, systemic inflammation due to infection; Renal (damage to the kidney itself): toxins or medication (e.g. some NSAIDs, aminoglycoside antibiotics, iodinated contrast, lithium); rhabdomyolysis (breakdown of muscle tissue)—the resultant release of myoglobin in the blood affects the kidney; it can be caused by injury (especially crush injury and extensive blunt trauma), statins, stimulants and some other drugs; hemolysis (breakdown of red blood cells)—the hemoglobin damages the tubules; it may be caused by various conditions such as sickle-cell disease, and lupus erythematosus; multiple myeloma, either due to hypercalcemia or “cast nephropathy” (multiple myeloma can also cause chronic renal failure by a different mechanism); Post-renal (obstructive causes in the urinary tract) due to: medication interfering with normal bladder emptying; benign prostatic hypertrophy or prostate cancer; kidney stones; abdominal malignancy (e.g. ovarian cancer, colorectal cancer); or obstructed urinary catheter. In some embodiments, the subject has at least one or more of the previously described conditions. Typical patients for which the prevention of AKI is desirable include cardiac/vascular surgery patients, renal transplant patients, and radiocontrast or chemotherapy patients. Typical patients for which the treatment of AKI is desirable include post-surgical patients, septic patients and patients with nephrotoxicity as a result of, for example, radiocontrast dyes, chemotherapy, and other toxins.

In certain embodiments, the subject does not have AKI as relating to a glomerular disease. In specific embodiments, the subject does not have glomerulonephritis. Even further specific embodiments, the subject does not have rapidly progressive glomerulonephritis (RPGN). In some cases, RPGN may be characterized by glomerular crescents on biopsy. In certain embodiments, subjects do not have acute glomerulonephritis related to anti-glomerular basement membrane disease/Goodpasture's syndrome, Wegener's granulomatosis or acute lupus nephritis with systemic lupus erythematosus.

It is recognized that in addition to acute kidney injury, the kidney injury in the subject may be chronic. In general, the methods described herein for prevention or therapeutic treatment of AKI may also be employed with respect to its chronic counterpart. Thus, the subject may have a kidney injury that is serious, but long-lasting and ongoing therapy using the methods of the invention would be beneficial. Similarly, the onset of such chronic conditions may be hampered by the treatment methods of the invention.

As used in the present application, “treat,” “treatment,” or “treating” is intended to refer to therapy or the complete or partial amelioration of a disease or condition, unless a different meaning is clearly intended from the context. Thus, “treating” a subject for kidney injury, either acute or chronic, refers to ameliorating the symptoms of a disorder or condition that is already present. As used in the present application, “prevent,” “prevention,” or “preventing” acute or chronic kidney injury is intended to refer to the complete or partial prophylaxis of the condition. “Prevention” also includes delaying the onset of the disorder or condition.

Similarly, “therapeutic treatment” does not require complete obliteration of the disorder or condition, but rather includes, as well, amelioration of symptoms, reduction in severity, and/or shortening the duration of its presence.

In certain embodiments of the present invention, the CXCR4 antagonists are compounds of formula (1)

Z-linker-Z′ (1)

or pharmaceutically acceptable salt or prodrug form thereof

wherein Z is a cyclic polyamine containing 9-32 ring members of which 2-8 are nitrogen atoms, said nitrogen atoms separated from each other by at least 2 carbon atoms, and wherein said heterocycle may optionally contain additional heteroatoms besides nitrogen and/or may be fused to an additional ring system;

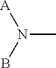

or Z is of the formula

wherein A comprises a monocyclic or bicyclic fused ring system containing at least one N and B is H or an organic moiety of 1-20 atoms,

Z′ may be embodied in a form as defined by Z above, or alternatively may be of the formula

—N(R)—(CR₂)ₙ—X

wherein each R is independently H or straight, branched or cyclic alkyl (1-6C), n is 1 or 2, and X is an aromatic ring, including heteroaromatic rings, or is a mercaptan or Z′ may be

—Ar(Y)ⱼ;

wherein Ar is an aromatic or heteroaromatic moiety, and each Y is independently a non-interfering substituent and j is 0-3; and

“linker” represents a bond, alkylene (1-6C) or may comprise aryl, fused aryl, oxygen atoms contained in an alkylene chain, or may contain keto groups or nitrogen or sulfur atoms;

in an amount effective to ameliorate or prevent acute kidney injury or a chronic form thereof in said subject.

In some embodiments, the compounds include those of formula (1) wherein formula (1) is in the form of its acid addition salt, or a prodrug. In specific embodiments, the acid addition salt is a hydrochloride salt.

Forms of the linker moiety include those wherein the linker is a bond, or wherein the linker includes an aromatic moiety flanked by alkylene, preferably methylene moieties Linking groups include the methylene bracketed forms of 1,3-phenylene, 2,6-pyridine, 3,5-pyridine, 2,5-thiophene, 4,4′-(2,2′-bipyrimidine); 2,9-(1,10-phenanthroline) and the like. One linker is 1,4-phenylene-bis-(methylene).

In some embodiments, the compounds include those of formula (1) wherein Z′ is

—N(R)—(CR₂)ₙ—X

wherein each R is independently H or straight, branched or cyclic alkyl (1-6C), n is 1 or 2, and X is an aromatic ring, including heteroaromatic rings, or is a mercaptan or Z′ may be

Ar(Y)ⱼ;

wherein Ar is an aromatic or heteroaromatic moiety, and each Y is independently a non-interfering substituent and j is 0-3.

In some embodiments, these compounds include those of above wherein Ar(Y)ⱼ is 2-aminomethyl-pyridine.

Some embodiments of Z and Z′ are cyclic polyamine moieties having from 9-24C that include 3-5 nitrogen atoms. Some specific embodiments are 1,5,9,13-tetraazacyclohexadecane; 1,5,8,11,14-pentaazacyclohexadecane; 1,4,8,11-tetraazacylotetradecane; 1,5,9-triazacyclododecane; 1,4,7,10-tetraazacyclododecane; and the like, including such cyclic polyamines which are fused to an additional aromatic or heteroaromatic rings and/or containing a heteroatom other than nitrogen incorporated in the ring. Embodiments wherein the cyclic polyamine contains a fused additional cyclic system or one or more additional heteroatoms are described in U.S. Pat. No. 5,698,546 and WO 01/44229 incorporated hereinabove by reference. Such embodiments include
3,7,11,17-tetraazabicyclo(13.3.1)heptadeca-1(17),13,15-triene;
4,7,10,17-tetraazabicyclo(13.3.1)heptadeca-1(17),13,15-triene;
1,4,7,10-tetraazacyclotetradecane; 1,4,7-triazacyclotetradecane; and
4,7,10-triazabicyclo(13.3.1)heptadeca-1(17),13,15-triene.

In some embodiments, the compounds include those of formula (1) wherein Z and Z′ are both cyclic polyamines. In some embodiments, Z and Z are identical. In further embodiments, Z is a cyclic polyamine that contains 10-24 members and contains 4 nitrogen atoms. In specific embodiments, Z and Z are both 1,4,8,11-tetraazocyclotetradecane.

In some embodiments, the compounds include those of formula (1) wherein the linker comprises an aromatic ring bracketed by two methylene moieties. In specific embodiments, the linker is 1,4-phenylene-bis-methylene.

In further specific embodiments, the compound of formula (1) is 1,1′-[1,4-phenylene-bis-(methylene)-bis-1,4,8,11-tetraazacyclotetradecane (plerixafor f/k/a AMD3100) or a pharmaceutically acceptable salt thereof. In other embodiments, the compound is N-[1,4,8,11-tetraazacyclotetradecanyl-1,4-phenylene-bis-(methylene)]-2-aminomethyl-pyridine (AMD3465) or a pharmaceutically acceptable salt thereof.

When Z′ is other than a cyclic polyamine as defined in Z, its embodiments include those set forth in U.S. Pat. No. 5,817,807, also incorporated herein by reference.

Forms wherein

Z is of the formula

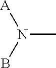

wherein A comprises a monocyclic or bicyclic fused ring system containing at least one N and B is H or an organic moiety of 1-20 atoms are disclosed in WO 00/56729; WO 02/22600; WO 02/34745; and WO 02/22599 all incorporated herein by reference.

In certain embodiments, the CXCR4 antagonists useful in the invention include those of formula (2)

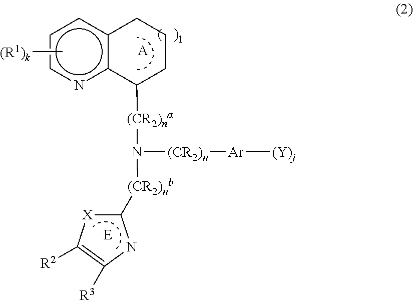

or pharmaceutically acceptable salt or prodrug form thereof

wherein:

Ring A optionally comprises a heteroatom selected from N, O and S;

the dotted lines represent optional unsaturation;

R¹, R² and R³ are non-interfering substituents;

k is 0-4;

1 is 0, 1, or 2;

X is unsubstituted or substituted C or N; or is O or S;

Ar is the residue of an aromatic or heteroaromatic moiety;

each n is independently 0-2;

each R is independently H or alkyl (1-6C);

j is 0-3; and

each Y is independently halo, OH, SH, SO, SO₂, or an organic moiety of 1-20C atoms that does not contain N wherein two such Y may be connected to form a fused ring with Ar, or is selected from the group consisting of

(CR₂)ₘCN,

—(CR₂)ₘNR⁵₂,

—(CR₂)ₘNR(CR₂)ₘNRR⁴,

—(CR₂)ₘNR(CR₂)ₘNR(CR₂)mNR⁵₂,

—(CR₂)ₘCO(CR₂)ₘNR⁵₂,

—(CR₂)ₘCO(CR₂)ₘNR(CR₂)ₘNRR⁴,

—(CR₂)ₘCO(CR₂)ₘNR(CR₂)ₘNR(CR₂)ₘNR⁵₂,

—(CR₂)ₘNRCO(CR₂)ₘNRR⁴,

—(CR₂)ₘNRCO(CR₂)ₘNR(CR₂)ₘNR⁵₂,

—(CR₂)ₘNRCO(CR₂)ₘNR(CR₂)ₘNR(CR₂)ₘNR(CR₂)ₘNR⁵₂,

—CH═N—Z″,

—(CR₂)ₘZ″,

NR(CR₂)ₘZ″,

—(CR₂)ₘNROH,

(CR₂)ₘCONROH, and

(CR₂)ₘCR═NOH,

wherein Z″ is an optionally substituted aromatic or heteroaromatic moiety containing 5-12 ring members; and

wherein R is as defined above, each m is independently 0-4, and R⁴ and each R⁵ is independently H, alkyl (1-6C), alkenyl (1-6C), alkynyl (1-6C), or acyl (1-6C), each optionally substituted by one or more nonaromatic, nonheterocyclic substituent(s), and wherein two R⁵ may be connected to form a cyclic amine, optionally containing one or more additional heteroatoms selected from N, O, and S.

In some embodiments, the compounds include those of formula (2) wherein ring E is coupled to the remainder of the molecule at position 2.

In some embodiments, R² and R³ taken together form a benzo substituent.

In certain embodiments, X is N and ring E comprises a pi bond coupled to one N.

In other embodiments, ring A is saturated and 1 is 1. In some embodiments, k is 0-1. In yet other embodiments, the ring system which includes A is tetrahydroquinoline or a substituted form thereof.

In some embodiments, the compounds include those of formula (2) wherein one of (CR₂)^{a}ₙ, and (CR₂)^{b}ₙ is CH₂ and the other is a bond. In other embodiments, (CR₂)^{a}ₙ is a bond and (CR₂)^{b}ₙ is CH₂. In some embodiments, at least one Y is —CH₂NH₂.

In certain embodiments, Ar is the residue of benzene, benzimidazole, benzothiazole, imidazole, oxazole, benztriazole, thiazole, pyridine, or pyrimidine.

Ar is the residue of an aromatic or heteroaromatic moiety which contains a single or fused ring system and containing 5-6 ring members in the monocyclic system and 9-12 members in the fused ring system. The residue may be optionally substituted. Examples of optionally substituted aromatic and heteroaromatic groups include benzene, naphthalene, dihydronaphthalene, tetrahydronaphthalene, pyridine, quinoline, isoquinoline, imidazole, benzimidazole, azabenzimidazole, benzotriazole, furan, benzofuran, thiazole, benzothiazole, oxazole, benzoxazole, pyrrole, indole, imidazole, tetrahydroquinoline, tetrahydroisoquinoline, pyrazole, thiophene, isoxazole, isothiazole, triazole, tetrazole, oxadiazole, thiadiazole, imidazoline, and benzopyran. Oxides of the nitrogen and sulfur containing heteroaromatic rings are also included in the present invention. In some embodiments Ar is phenylene, pyridylene or pyridnylene.

When compounds of formula (2) are substituted by non-interfering substituents or contain elements that are “optionally substituted” these substituents may include halogen, nitro, cyano, carboxylic acid, optionally substituted alkyl, alkenyl or cycloalkyl groups, an optionally substituted hydroxyl group, an optionally substituted thiol group, an optionally substituted amino, an optionally substitute acyl group, an optionally substituted carboxylate, carbamate, carboxamide or sulfonamide group, or an optionally substituted aromatic or heterocyclic group.

Examples of halogen include fluorine, chlorine, bromine and iodine.

Examples of optionally substituted alkyl include C₁₋₁₀ alkyl, including methyl, ethyl propyl, etc.; examples of optionally substituted alkenyl groups include C₂₋₁₀ alkenyl such as allyl, crotyl, 2-pentenyl, 3-hexenyl, etc.; and examples of optionally substituted cycloalkyl groups include C₃₋₁₀ cycloalkyl such as cyclopropyl, cyclobutyl, cyclopentyl, cyclohexyl, cycloheptyl, etc. In these cases, C₁₋₆ alkyl, alkenyl and cycloalkyl are preferred. The optional substituent may also be an optionally substituted aralkyl (e.g., phenyl C₁₋₄ alkyl) or heteroalkyl for example, phenylmethyl (benzyl), phenylethyl, pyridinylmethy, pyridinylethyl, etc. The heterocyclic group may be a 5 or 6 membered ring containing 1-4 heteroatoms.

Examples of optionally substituted hydroxyl and thiol groups include those wherein the substituent is an optionally substituted alkyl (e.g., C₁₋₁₀ alkyl) such as methyl, ethyl, propyl, isopropyl, butyl, isobutyl, sec-butyl, tert-butyl, pentyl, etc., preferably (C₁₋₆) alkyl; an optionally substituted cycloalkyl (e.g., C₃₋₇ cycloalkyl, etc., such as cyclopropyl, cyclobutyl, cyclopentyl, cyclohexyl, cycloheptyl, etc.); an optionally substituted aralkyl (e.g., phenyl-C₁₋₄ alkyl, e.g., benzyl, phenethyl, etc.). Where there are two adjacent hydroxyl or thiol substituents, the heteroatoms may be connected via an alkylene group such as O(CH₂)ₙO and S(CH₂)ₙS (where n=1-5). Examples include methylenedioxy, ethylenedioxy, etc. Oxides of thio-ether groups such as sulfoxides and sulfones are also encompassed.

Further examples of the optionally substituted hydroxyl group include an optionally substituted C₂₋₄ alkanoyl (e.g., acetyl, propionyl, butyryl, isobutyryl, etc.), C₁₋₄ alkylsulfonyl (e.g., methanesulfonyl, ethanesulfonyl, etc.) and an optionally substituted aromatic and heterocyclic carbonyl group including benzoyl, pyridinecarbonyl, etc.

The substituents on optionally substituted amino group may bind to each other to form a cyclic amino group (e.g., 5- to 6-membered cyclic amino, etc., such as tetrahydropyrrole, piperazine, piperidine, pyrrolidine, morpholine, thiomorpholine, pyrrole, imidazole, etc.). Said cyclic amino group may have a substituent, and examples of the substituents include halogen (e.g., fluorine, chlorine, bromine, iodine, etc.), nitro, cyano, hydroxy group, thiol group, amino group, carboxyl group, an optionally halogenated C₁₋₄ alkyl (e.g., trifluoromethyl, methyl, ethyl, etc.), an optionally halogenated C₁₋₄ alkoxy (e.g., methoxy, ethoxy, trifluoromethoxy, trifluoroethoxy, etc.), C₂₋₄ alkanoyl (e.g., acetyl, propionyl, etc.), C₁₋₄ alkylsulfonyl (e.g., methanesulfonyl, ethanesulfonyl, etc.) the number of preferred substituents are 1 to 3.

The amino group may also be substituted once or twice (to form a secondary or tertiary amine) with a group such as an optionally substituted alkyl group including C₁₋₁₀ alkyl (e.g., methyl, ethyl propyl, etc.); an optionally substituted alkenyl group such as allyl, crotyl, 2-pentenyl, 3-hexenyl, etc., or an optionally substituted cycloalkyl group such as cyclopropyl, cyclobutyl, cyclopentyl, cyclohexyl, cycloheptyl, etc. In these cases, C₁₋₆ alkyl, alkenyl and cycloalkyl are preferred. The amine group may also be optionally substituted with an aromatic or heterocyclic group, aralkyl (e.g., phenyl C₁₋₄ alkyl) or heteroalkyl for example, phenyl, pyridine, phenylmethyl (benzyl), phenethyl, pyridinylmethyl, pyridinylethyl, etc. The heterocyclic group may be a 5 or 6 membered ring containing 1-4 heteroatoms. The optional substituents of the “optionally substituted amino groups are the same as defined above for the “optionally substituted cyclic amino group.”

The amino group may be substituted with an optionally substituted C₂₋₄ alkanoyl, e.g., acetyl, propionyl, butyryl, isobutyryl etc., or a C₁₋₄ alkylsulfonyl (e.g., methanesulfonyl, ethanesulfonyl, etc.) or a carbonyl or sulfonyl substituted aromatic or heterocyclic ring, e.g., benzenesulfonyl, benzoyl, pyridinesulfonyl, pyridinecarbonyl, etc. The heterocycles are as defined above.

Examples of the optionally substituted acyl groups include a carbonyl group or a sulfinyl or sulfonyl group binding to hydrogen; or to an optionally substituted alkyl (e.g., C₁₋₁₀ alkyl such as methyl, ethyl, propyl, isopropyl, butyl, isobutyl, sec-butyl, tert-butyl, pentyl, isopentyl, neopentyl, hexyl, heptyl, octyl, nonyl, decyl, etc., preferably lower (C₁₋₆) alkyl, etc.; an optionally substituted cycloalkyl (e.g., C₃₋₇ cycloalkyl, etc., such as cyclopropyl, cyclobutyl, cyclopentyl, cyclohexyl, cycloheptyl, etc.); an optionally substituted alkenyl (e.g., C₂₋₁₀ alkenyl such as allyl, crotyl, 2-pentenyl, etc., preferably lower (C₂₋₆) alkenyl, etc.); an optionally substituted cycloalkenyl (e.g., C₃₋₇ cycloalkenyl, etc., such as 2-cyclopentenyl, 2-cyclohexenyl, 2-cyclopentenylmethyl, 2-cyclohexenylmethyl, etc.) an optionally substituted 5- to 6-membered monocyclic aromatic group (e.g., phenyl, pyridyl, etc.).

Examples of the optionally substituted carboxylate group (ester groups) include an optionally substituted alkyl (e.g., C₁₋₁₀ alkyl such as methyl, ethyl, propyl, isopropyl, butyl, isobutyl, sec-butyl, tert-butyl, pentyl, isopentyl, neopentyl, hexyl, heptyl, octyl, nonyl, decyl, etc., preferably lower (C₁₋₆) alkyl, etc.); an optionally substituted cycloalkyl, e.g., C₃₋₇ cycloalkyl, etc. such as cyclopropyl, cyclobutyl, cyclopentyl, cyclohexyl, cycloheptyl, etc.); an optionally substituted alkenyl (e.g., C₂₋₁₀ alkenyl such as allyl, crotyl, 2-pentenyl, 3-hexenyl, etc., preferably lower (C₂₋₆) alkenyl, etc.); an optionally substituted cycloalkenyl e.g., C₃₋₇ cycloalkenyl, etc., such as 2-cyclohexenylmethyl, etc.); an optionally substituted aryl e.g., phenyl, naphthyl, etc.) and C₁₋₄ aryl for example, benzyl, phenethyl etc. Groups such as methoxymethyl, methoxyethyl, etc., are also encompassed.

Examples of the optionally substituted carboxamide and sulfonamide groups are identical in terms of the amine definition as the “optionally substituted amino group” defined above.

Examples of the optionally substituted aromatic or heterocyclic groups are phenyl, naphthyl, or a 5- or 6-membered heterocyclic ring containing 1-4 heteroatoms. The optional substituents are essentially identical to those listed above.

The non-interfering substituents R¹, R² and R³ are similar to those set forth as “optional substituents”. R¹ can be selected from the optional substituents set forth above, preferably halo, substituted or unsubstituted alkyl, substituted or unsubstituted hydroxyl, substituted or unsubstituted amino, substituted or unsubstituted thiol, and substituted or unsubstituted acyl. Preferably k is 0-2, preferably 0-1, and more preferably 0.

The substituents R² and R³ may be selected from the preferred embodiments of R¹ listed immediately above, or may be joined to form a saturated or unsaturated ring system, preferably a benzo ring system.

In the above formula 2, examples of the optionally substituted ring system containing ring A are dihydroquinoline, tetrahydroquinoline, pyranopyridine, dihydropyranopyridine, thiapyranopyridine, dihydrothiapyranopyridine, dihydronaphthyridine, tetrahydronaphthyridine. Oxides of sulfur-containing heterocycles are also encompassed in the present invention. In the above ring system containing Ring A, the optional nitrogen atom may be substituted with hydrogen, a substituted alkyl, alkenyl, cycloalkyl or aryl group, or may be the nitrogen atom of a carboxamide, carbamate or sulfonamide. If 1 is 1=1, ring A may be saturated. In one embodiment, A is tetrahydroquinoline.

In the above formula 2, X may be CH (pyrrole), O (oxazole), S (thiazole), NH or NR (imidazole) where R is a C₁₋₆ alkyl group or acyl, sulfonyl group. Two adjacent R¹ and/or R² and R³ may be joined to form an optionally substituted, fused 5-7 membered ring. Examples of fused ring systems include but are not limited to indole, tetrahydroindole, benzimidazole, tetrahydrobenzimidazole, azabenzimidazole, benzoxazole, tetrahydrobenzoxazole, benzothiazole, tetrahydrobenzothiazole. The ring systems resulting from R² and R³ include those which result in benzothiazole and benzoimidazole.

In the compounds of formula 2, one of the (CR₂)ₙ linkers between the ring system containing ring A and ring E may be that wherein n is 0, i.e., the linkage is merely a covalent bond. (CR₂)ₙ in this context may also be ethylene or methylene. The linkage between the nitrogen shown in formula 2 and ring A may be a bond. As shown, ring E may be coupled to the linker through any position, including position 2, 4 or 5.

In the compounds of formula 2, values of j may be 0, 1 or 2. The embodiments of Y may be varied widely provided Y does not contain nitrogen. Thus, Y may be halo, OH, SH, SO, SO₂ and the like, or a substituent of 1-20 carbons, optionally containing as a substitution, for one or more said carbons, a heteroatom such as O or S. Embodiments wherein N is not present in Y include halo, optionally substituted alkyl, optionally substituted hydroxyl, optionally substituted thiol, and optionally substituted carboxylate, and a saturated or unsaturated ring. These substituents are described above. Where N is included in Y, Y is selected from the moieties set forth hereinabove. In these substituents, Z″ is an aromatic or heteroaromatic moiety containing 5-12 ring members. Thus, Y may include a single or fused ring. Examples of Z″ are identical to those set forth with regard to the aromatic residue Ar set forth above, but are monovalent.

As shown, in certain embodiments, R, defined as H or alkyl (1-6C), is replaced by R⁴ or R⁵ which have a broader definitions and can include the embodiments of R as well as embodying optionally substituted alkenyl, acyl, and the like as set forth above. Forms of R⁴ and R⁵ include those typified by R and optionally substituted alkenyl. Embodiments where two R⁵ are connected to form a cyclic amine, including those which contain one or more additional heteroatoms such as N, O, and/or S, are also included.

Forms of Y when Y contains N are those wherein R is in all cases H or methyl, preferably H and those where two R⁵ are coupled. Exemplified are those of the formula

—(CR₂)ₘCN,

—(CR₂)ₘNR⁵₂,

—(CR₂)ₘNR(CR₂)ₘNRR⁴,

—(CR₂)ₘCO(CR₂)ₘNR⁵₂,

—(CR₂)ₘZ″, and

—NR(CR₂)ₘZ″,

and those wherein Y comprises guanidino or NHNHR,

wherein (CR₂)ₘ may be CH₂, CH₂CH₂, or CH₂CH₂CH₂, or wherein m is 0, and those wherein R⁴ or R⁵ is H or may be lower alkyl, alkenyl, or hydrogen, or wherein both R⁵ are identical.

Forms wherein Y is —CH₂NH₂, CH₂CH₂NH₂, —CH₂NMe₂, —CH₂CH₂NMe₂, —CONH₂, —CONMe₂, and the like are included.

Z″ can be optionally substituted residues of benzene, oxazole, imidazole, thiazole, benzimidazole, benzthiazole, benzoxazole, indole, thiophene, tetrazine, pyrimidine, pyridine, and the like.

In certain embodiments, the CXCR4 antagonists useful in the invention include those of formula (3)

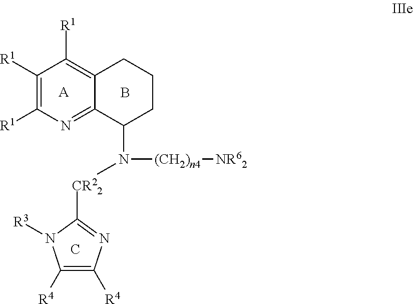

or a pharmaceutically acceptable salt thereof; and including any stereoisomeric forms thereof;

wherein n4 is 2-4;

each R¹ is independently H, halo, alkyl, alkoxy, or CF₃;

each R² is independently H or alkyl;

R³ is H, alkyl, alkenyl, arylalkyl, or aryl;

each R⁴ is independently H or alkyl, or the two R⁴ groups may be taken together with the ring to which they are attached to form an optionally substituted 6-membered aromatic or heteroaromatic ring; and

each R⁶ is independently H, arylalkyl, acyl, arylacyl, or arylsulfonyl, wherein the aryl moieties thereof optionally contain one or more heteroatoms selected from the group consisting of O, S, and N.

Included are those wherein each R¹ is H; and/or

wherein each R² is H; and/or

wherein each R³ is H, and/or

wherein the two R⁴ groups may be taken together with the ring to which they are attached to form an optionally substituted phenyl ring; and/or

wherein each R⁶ is H.

In certain embodiments, the CXCR4 antagonists suitable for the methods disclosed herein are of the formula

V—CR¹R²—Ar—CR³R⁴—N(R⁵)—(CR⁶R⁷)ₓ—R⁸)

wherein V is a 1,4,8,11-tetraazacyclotetra-decanyl group;

R¹ to R⁷ may be the same or different and are independently selected from hydrogen or straight, branched or cyclic C₁₋₆ alkyl;

R⁸ is a pyridine, pyrimidine, pyrazine, imidazole, thiophene, thiophenyl, aminobenzyl, piperidinyl, piperazinyl group, or a mercaptan group;

Ar is a phenylene ring optionally substituted with an electron donating or withdrawing group selected from the group consisting of alkyl, aryl, amino, alkoxy, hydroxy, halogen, carboxyl and carboxamido;

x is 1 or 2;

or a pharmaceutically acceptable salt thereof.

In certain embodiments, the CXCR4 antagonists suitable for the methods disclosed herein are

of the formula

V—CR¹R²—Ar—CR³R⁴—N(R⁵)—(CR⁶R⁷)ₓ—R⁸

wherein V is an optionally substituted 4,7,10,17-tetraazabicyclo[13.3.1]heptadeca-1(17),13,15-trienyl system;

R¹ to R⁷ may be the same or different and are independently selected from hydrogen or straight, branched or cyclic C₁₋₆ alkyl;

R⁸ is pyridyl, pyrimidinyl, pyrazinyl, imidazolyl, thiophene-yl, thiophenyl, aminobenzyl, piperidinyl, piperazinyl or mercaptan;

Ar is a phenylene ring optionally substituted at single or multiple positions with alkyl, aryl, amino, alkoxy, hydroxy, halogen, carboxyl and/or carboxamido; and

x is 1 or 2;

or a pharmaceutically acceptable salt thereof.

In certain embodiments, the CXCR4 antagonists suitable for the methods disclosed herein are of the formula

V—CR¹R²—Ar—CR³R⁴—N(R⁵)—(CR⁶R⁷)ₓ—R⁸

wherein V is an optionally substituted 1,4,7-triazacyclotetra-decanyl or a 4,7,10-triazabicyclo[13.3.1]heptadeca-1(17),13,15-trienyl system, optionally substituted by hydroxyl, alkoxy, thiol, thioalkyl, halogen, nitro, carboxy, carboxamido, sulfonate and/or phosphate;

R¹ to R⁷ may be the same or different and are independently selected from hydrogen or straight, branched or cyclic C₁₋₆ alkyl;

R⁸ is pyridyl, pyrimidinyl, pyrazinyl, imidazolyl, thiophene-yl, thiophenyl, aminobenzyl, piperidinyl, piperazinyl or mercaptan;

Ar is a phenylene ring optionally substituted at single or multiple positions with alkyl, aryl, amino, alkoxy, hydroxy, halogen, carboxyl and/or carboxamido; and

x is 1 or 2;

or a pharmaceutically acceptable salt thereof.

In certain embodiments, the CXCR4 antagonists suitable for the methods disclosed herein are of the formula

V—CR¹R²—Ar—CR³R⁴—N(R⁵)—(CR⁶R⁷)ₓ—R⁸

wherein V is a 1,4,8,11-tetraazacyclotetradecanyl group, and which may optionally comprise a fused aromatic or heteroaromatic ring;

R¹ to R⁷ are independently hydrogen, or straight, branched or cyclic C₁₋₆ alkyl;

R⁸ is a heterocyclic group, a substituted aromatic group, or a mercaptan group;

Ar is an aromatic ring or heteroaromatic ring each said ring being optionally substituted;

x is 1 or 2;

or the acid addition salts and metal complexes thereof; or may be

of the formula

Z—R-A—R¹—Y

in which Z and Y are identical cyclic polyamine moieties having from 10 to 15 ring members and from 3 to 6 amine nitrogens in the ring spaced by 2 or more carbon atoms from each other, said amine nitrogens being the only ring heteroatoms,

A is an aromatic or heteroaromatic moiety other than quinoline,

R and R¹ are each methylene linked to nitrogen atoms in Z and Y,

the amine nitrogen atoms being otherwise unsubstituted, or a pharmaceutically acceptable salt thereof.

Embodiments of the compound of the formula (1) include 2,2′-bicyclam; 6,6′-bicyclam; the embodiments set forth in U.S. Pat. Nos. 5,021,409, and 6,001,826, and in particular 1,1′-[1,4-phenylene-bis(methylene)]-bis-1,4,8,11-tetraazacyclotetradecane, set forth in U.S. Pat. No. 5,583,131, and designated herein AMD3100, and N[1,4,8,11-tetraazacyclotetra-decanyl-1,4-phenylene-bis-(methylene)]-2-aminomethyl-pyridine (AMD3465).

Methods to synthesize the compounds useful in the method of the invention are set forth in the U.S. patents incorporated hereinabove by reference.

The compounds of the invention may be prepared in the form of prodrugs, i.e., protected forms which release the compounds of the invention after administration to the subject. Typically, the protecting groups are hydrolyzed in body fluids such as in the bloodstream thus releasing the active compound or are oxidized or reduced in vivo to release the active compound. A discussion of prodrugs is found in Smith and Williams Introduction to the Principles of Drug Design, Smith, H. J.; Wright, 2^{nd} ed., London (1988).

The compounds of the invention, as they are polyamines, may be administered prepared in the forms of their acid addition salts or metal complexes thereof. Suitable acid addition salts include salts of inorganic acids that are biocompatible, including HCl, HBr, sulfuric, phosphoric and the like, as well as organic acids such as acetic, propionic, butyric and the like, as well as acids containing more than one carboxyl group, such as oxalic, glutaric, adipic and the like. Typically, at physiological pH, the compounds of the invention will be in the forms of the acid addition salts. In addition, when prepared as purified forms, the compounds may also be crystallized as the hydrates.

The compounds of the invention may be administered as sole active ingredients, as mixtures of various compounds of formula (1), and/or in admixture with additional active ingredients that are therapeutically or nutritionally useful, such as antibiotics, vitamins, herbal extracts, anti-inflammatories, glucose, antipyretics, analgesics, granulocyte-macrophage colony stimulating factor (GM-CSF), Interleukin-1 (IL-1), Interleukin-3 (IL-3), Interleukin-8 (IL-8), PIXY-321 (GM-CSF/IL-3 fusion protein), macrophage inflammatory protein, stem cell factor, thrombopoietin, growth related oncogene or chemotherapy and the like.

The compounds of the invention may be formulated for administration to animal subject using commonly understood formulation techniques well known in the art. Formulations which are suitable for particular modes of administration and for compounds of the type disclosed herein may be found in Remington's Pharmaceutical Sciences, latest edition, Mack Publishing Company, Easton, Pa.

The compounds may be administered by injection, such as by intravenous injection, but also by subcutaneous or intraperitoneal injection, and the like. Additional parenteral routes of administration include intramuscular and intraarticular injection. For intravenous or parenteral administration, the compounds are formulated in suitable liquid form with excipients as required. The compositions may contain liposomes or other suitable carriers. For injection intravenously, the solution is made isotonic using standard preparations such as Hank's solution.

Besides injection, other routes of administration may also be used. The compounds may be formulated into tablets, capsules, syrups, powders, or other suitable forms for administration orally. By using suitable excipients, these compounds may also be administered through the mucosa using suppositories or intranasal sprays. Transdermal administration can also be effected by using suitable penetrants and controlling the rate of release.

The formulation and route of administration chosen will be tailored to the individual subject, the nature of the condition to be treated in the subject, and generally, the judgment of the attending practitioner.

Suitable dosage ranges for the compounds disclosed herein vary according to these considerations, but in general, the compounds are administered in the range of about 0.1 μg/kg-5 mg/kg of body weight; preferably the range is about 1 μg/kg-300 μg/kg of body weight; more preferably about 10 μg/kg-100 μg/kg of body weight. In certain embodiments, a compound of the invention is administered in an amount of 240 μg/kg of body weight. For a typical 70-kg human subject, thus, the dosage range is from about 0.7 μg-350 mg; preferably about 700 μg-21 mg; most preferably about 700 μg-7 mg. Dosages may be higher when the compounds are administered orally or transdermally as compared to, for example, i.v. administration.

In some embodiments, the compound of disclosed herein are administered to said subject in the dosage range of about 0.1 μg/kg-5 mg/kg of body weight. In some embodiments, the subject is human.

The compounds may be administered as a single bolus dose, a dose over time, as in i.v. or transdermal administration, or in multiple dosages.

Subjects that will respond favorably to the method of the invention include medical and veterinary subjects generally, including human patients. Among other subjects for whom the methods of the invention is useful are cats, dogs, large animals, avians such as chickens, and the like. In general, any subject who would benefit from an elevation of progenitor cells and/or stem cells, or whose progenitor cells and/or stem cells are desirable for stem cell transplantation are appropriate for administration of the invention method.

In some embodiments, subjects that are at risk of AKI are transplant recipients. Transplanted material, as used herein, includes cells, tissue, grafts, fluids, and organs, and can originate from living or deceased organisms. In particular embodiments, the transplanted material is a highly vascularized tissue or organ. In even more specific embodiments, the transplant is kidney or liver. In alternative embodiments, the transplant is selected from the group consisting of blood, heart, skin, bone marrow, endothelial cells, lung, pancreas, intestine, penis, bone, tendons, heart valve, veins, arm, hand and the cornea. In some embodiments, transplants include tissue-engineered constructs that are composed generally of a biological or synthetic matrix containing cells, which may also include various therapeutic agents and growth factors. Transplants may include autografts, allografts, heterogenic transplants or a combination thereof.

Various optional constituents, such as immunosuppressive agents, growth factors and other substances, can also be included with the endothelial cells and/or the transplant. Such constituents include, inter alia, extracellular matrix proteins such as collagen and fibronectin; integrins; growth factors such as tissue growth factors, etc. In particular, angiogenic factors can be administered along with the transplant, which include basic fibroblast growth factor, acidic fibroblast growth factor, endothelial cell growth factor, angiogenin, and transforming growth factors alpha and beta.

In general, the compounds useful in the invention will have the profile shown in Table 2:

In certain embodiments, specific CXCR4 antagonists suitable for the methods disclosed herein are selected from the following compounds including their salts:
N-[1,4,8,11-tetraazacyclotetradecanyl-1,4-phenylenebis(methylene)]-2-(aminomethyl)pyridine (AMD3465);
7,7′-[1,4-phenylenebis(methylene)]bis-4,7,10,17-tetraazabicyclo-[13.3.1]heptadeca-1(17),13,15-triene;
7,7′-[1,4-phenylenebis(methylene)]bis-3,7,11,17-tetraazabicyclo[13.3.1]heptadeca-1(17),13,15-triene;
1,1′-[1,3-phenylenebis(methylene)]-bis-1,4,8,11-tetra-azacyclotetradecane;
1,1′-[1,4-phenylenebis(methylene)]-bis-1,4,8,11-tetra-azacyclotetradecane (AMD3100);
1,1′-[1,4-phenylene-bis-(methylene)]-bis-1,4,7,10-tetraazacyclotetradecane;
1,1′-[1,3-phenylene-bis-(methylene)]-bis-1,4,7,10-tetraazacyclotetradecane;
11,11′-(1,2-propanediyl)bis-1,4,8,11-tetraazacyclotetradecane;
N-[4-(1,4,7-triazacyclotetra-decane)-1,4-phenylenebis(methylene)]-2-(aminomethyl)pyridine;
N-[7-(4,7,10-triazabicyclo[13.3.1]heptadeca-1(17),13,15-triene)-1,4-phenylenebis(methylene)]-2-(aminomethyl)pyridine;
N-[7-(4,7,10,17-tetraazabicyclo[13.3.1]heptadeca-1(17),13,15-triene)-1,4-phenylenebis(methylene)]-2-(aminomethyl)pyridine;
N-[4-[4,7,10,17-tetraazabicyclo[13.3.1]heptadeca-1(17),13,15-triene]-1,4-phenylenebis(methylene)]-2-(aminomethyl)pyridine;
3,3′-(bis-1,5,9,13-tetraazacyclohexadecane);
3,3′-(bis-1,5,8,11,14-pentaazacyclohexadecane), methylene (or polymethylene) di-1-N-1,4,8,11-tetraazacyclotetradecane;
3,3′-bis-1,5,9,13,-tetraazacyclohexadecane;
3,3′-bis-1,5,8,11,14-pentaazacyclohexadecane;
5,5′-bis-1,4,8,11-tetraazacyclotetradecane;
2,5′-bis-1,4,8,11-tetraazacyclotetradecane;
2,6′-bis-1,4,8,11-tetraazacyclotetradecane;
11,11′-(1,2-ethanediyl)bis-1,4,8,11-tetraazacyclotetradecane;
11,11′-(1,2-propanediyl)bis-1,4,8,11-tetraazacyclotetradecane;
11,11′-(1,2-butanediyl)bis-1,4,8,11-tetraazacyclotetradecane;
11,11′-(1,2-pentanediyl)bis-1,4,8,11-tetraazacyclotetradecane;
11,11′-(1,2-hexanediyl)bis-1,4,8,11-tetraazacyclotetradecane;
3,3′-bis-1,5,9,13-tetraazacyclohexadecane;
3,3′-bis-1,5,8,11,14-pentaazacyclohexadecane;
5,5′-bis-1,4,8,11-tetraazacyclotetradecane;
2,5′-bis-1,4,8,11-tetraazacyclotetradecane;
2,6′-bis-1,4,8,11-tetraazacyclotetradecane;
11,11′-(1,2-ethanediyl)bis-1,4,8,11-tetraazacyclotetradecane;
11,11′-(1,2-propanediyl)bis-1,4,8,11-tetraazacyclotetradecane;
11,11′-(1,2-butanediyl)bis-1,4,8,11-tetraazacyclotetradecane;
11,11′-(1,2-pentanediyl)bis-1,4,8,11-tetraazacyclotetradecane;
11,11′-(1,2-hexanediyl)bis-1,4,8,11-tetraazacyclotetradecane;
1,1′-[1,3-phenylenebis(methylene)]-bis-1,4,8,11-tetra-azacyclotetradecane;
1,1′-[1,4-phenylenebis(methylene)]-bis-1,4,8,11-tetra-azacyclotetradecane;
1,1′-[3,3′-biphenylene-bis-(methylene)]-bis-1,4,8,11-tetraazacyclotetradecane;
11,11′-[1,4-phenylene-bis-(methylene)]-bis-1,4,7,11-tetraazacyclotetradecane;
1,11′-[1,4-phenylene-bis(methylene)]-1,4,8,11-tetraazacyclotetradecane;
1,1′-[2,6-pyridine-bis-(methylene)]-bis-1,4,8,11-tetraazacyclotetradecane;
1,1-[3,5-pyridine-bis-(methylene)]-bis-1,4,8,11-tetraazacyclotetradecane;
1,1′-[2,5-thiophene-bis-(methylene)]-bis-1,4,8,11-tetraazacyclotetradecane;
1,1′-[4,4′-(2,2′-bipyridine)-bis-(methylene)]-bis-1,4,8,11-tetraazacyclotetradecane;
1,1′-[2,9-(1,10-phenanthroline)-bis-(methylene)]-bis-1,4,8,11-tetraazacyclotetradecane;
1,1′-[1,3-phenylene-bis-(methylene)]-bis-1,4,7,10-tetraazacyclotetradecane;
1,1′-[1,4-phenylene-bis-(methylene)]-bis-1,4,7,10-tetraazacyclotetradecane;
1,1′-[5-nitro-1,3-phenylenebis(methylene)]bis-1,4,8,11-tetraazacyclotetradecane;
1,1′-[2,4,5,6-tetrachloro-1,3-phenylenebis(methylene)]bis-1,4,8,11-tetraazacyclotetradecane;
1,1′-[2,3,5,6-tetrafluoro-1,4-phenylenebis(methylene)]bis-1,4,8,11-tetraazacyclotetradecane;
1,1′-[1,4-naphthylene-bis-(methylene)]bis-1,4,8,11-tetraazacyclotetradecane;
1,1′-[1,3-phenylenebis-(methylene)]bis-1,5,9-triazacyclododecane;
1,1′-[1,4-phenylene-bis-(methylene)]-1,5,9-triazacyclododecane;
1,1′-[2,5-dimethyl-1,4-phenylenebis-(methylene)]-bis-1,4,8,11-tetraazacyclotetradecane;
1,1′-[2,5-dichloro-1,4-phenylenebis-(methylene)]-bis-1,4,8,11-tetraazacyclotetradecane;
1,1′-[2-bromo-1,4-phenylenebis-(methylene)]-bis-1,4,8,11-tetraazacyclotetradecane;
1,1′-[6-phenyl-2,4-pyridinebis-(methylene)]-bis-1,4,8,11-tetraazacyclotetradecane;
7,7′-[1,4-phenylene-bis(methylene)]bis-3,7,11,17-tetraazabicyclo[13.3.1]heptadeca-1(17),13,15-triene;
7,7′-[1,4-phenylene-bis(methylene)]bis[15-chloro-3,7,11,17-tetraazabicyclo[13.3.1]heptadeca-1(17),13,15-triene];
7,7′-[1,4-phenylene-bis(methylene)]bis[15-methoxy-3,7,11,17-tetraazabicyclo[13.3.1]heptadeca-1(17),13,15-triene];
7,7′-[1,4-phenylene-bis(methylene)]bis-3,7,11,17-tetraazabicyclo[13.3.1]-heptadeca-13,16-triene-15-one;
7,7′-[1,4-phenylene-bis(methylene)]bis-4,7,10,17-tetraazabicyclo[13.3.1]-heptadeca-1(17),13,15-triene;
8,8′-[1,4-phenylene-bis(methylene)]bis-4,8,12,19-tetraazabicyclo[15.3.1]nonadeca-1(19),15,17-triene;
6,6′-[1,4-phenylene-bis(methylene)]bis-3,6,9,15-tetraazabicyclo[11.3.1]pentadeca-1(15),11,13-triene;
6,6′-[1,3-phenylene-bis(methylene)]bis-3,6,9,15-tetraazabicyclo[11.3.1]pentadeca-1(15),11,13-triene;
17,17′-[1,4-phenylene-bis(methylene)]bis-3,6,14,17,23,24-hexaazatricyclo[17.3.1.1^{8,12}]tetracosa-1(23),8,10,12(24),19,21-hexaene;
N-[1,4,8,11-Tetraazacyclotetradecanyl-1,4-phenylenebis(methylene)]-2-(amino-methyl)pyridine;
N-[1,4,8,11-Tetraazacyclotetradecanyl-1,4-phenylenebis(methylene)]-N-methyl-2-(aminomethyl)pyridine;
N-[1,4,8,11-Tetraazacyclotetradecanyl-1,4-phenylenebis(methylene)]-4-(amino-methyl)pyridine;
N-[1,4,8,11-Tetraazacyclotetradecanyl-1,4-phenylenebis(methylene)]-3-(amino-methyl)pyridine;
N-[1,4,8,11-Tetraazacyclotetradecanyl-1,4-phenylenebis(methylene)]-(2-amino-methyl-5-methyl)pyrazine;
N-[1,4,8,11-Tetraazacyclotetradecanyl-1,4-phenylenebis(methylene)]-2-(amino-ethyl)pyridine;
N-[1,4,8,11-Tetraazacyclotetradecanyl-1,4-phenylenebis(methylene)]-2-(amino-methyl)thiophene;
N-[1,4,8,11-Tetraazacyclotetradecanyl-1,4-phenylenebis(methylene)]-2-(amino-ethyl)mercaptan;
N-[1,4,8,11-Tetraazacyclotetradecanyl-1,4-phenylenebis(methylene)]-2-amino-benzylamine;
N-[1,4,8,11-Tetraazacyclotetradecanyl-1,4-phenylenebis(methylene)]-4-amino-benzylamine;
N-[1,4,8,11-Tetraazacyclotetradecanyl-1,4-phenylenebis(methylene)]-4-(amino-ethyl)imidazole;
N-[1,4,8,11-Tetraazacyclotetradecanyl-1,4-phenylenebis(methylene)]-benzylamine;
N-[1,4,8,11-Tetraazacyclotetradecanyl-1,4-phenylenebis(methylene)]-purine;
N-[1,4,8,11-Tetraazacyclotetradecanyl-1,4-phenylenebis(methylene)]-4-phenylpiperazine;
N-[4-(1,4,7-Triazacyclotetra-decanyl)-1,4-phenylenebis(methylene)]-2-(aminomethyl)pyridine;
N-[7-(4,7,10,17-Tetraazabicyclo[13.3.1]heptadeca-1(17),13,15-trienyl)-1,4-phenylenebis(methylene)]-2-(aminomethyl)pyridine;
N-[7-(4,7,10-Triazabicyclo[13.3.1]heptadeca-1(17),13,15-trienyl)-1,4-phenylenebis(methylene)]-2-(aminomethyl)pyridine;
N-[4-[4,7,10-Triazabicyclo[13.3.1]heptadeca-1(17),13,15-trienyl]-1,4-phenylenebis(methylene)]-2-(aminomethyl)pyridine;
N-[1-(1,4,7-Triazacyclotetra-decanyl)-1,4-phenylenebis(methylene)]-2-(aminomethyl)pyridine;
N-[4-[4,7,10,17-Tetraazabicyclo[13.3.1]heptadeca-1(17),13,15-trienyl]-1,4-phenylenebis(methylene)]-2-(aminomethyl)pyridine;
N-[3-(3,6,17-Triazabicyclo[13.3.1]heptadeca-1(17),13,15-trienyl)-1,4-phenylenebis(methylene)]-2-(aminomethyl)pyridine;
N-[3-(3,6,17-Triazabicyclo[13.3.1]heptadeca-1(17),13,15-trienyl)-1,3-phenylenebis(methylene)]-2-(aminomethyl)pyridine;
N-[4-(4,7,17-Triazabicyclo[13.3.1]heptadeca-1(17),13,15-trienyl)-1,4-phenylenebis(methylene)]-2-(aminomethyl)pyridine;
N-[7-(4,7,17-Triazabicyclo[13.3.1]heptadeca-1(17),13,15-trienyl)-1,4-phenylenebis(methylene)]-2-(aminomethyl)pyridine;
N-[6-(3,6,9-Triazabicyclo[11.3.1]pentadeca-1(15),11,13-trienyl)-1,3-phenylenebis(methylene)]-2-(aminomethyl)pyridine;
N-[7-(4,10,17-Triazabicyclo[13.3.1]heptadeca-1(17),13,15-trienyl)-1,4-phenylenebis(methylene)]-2-(aminomethyl)pyridine;
N-[4-(1,7-Diazacyclotetradecanyl)-1,4-phenylenebis(methylene)]-2-(aminomethyl)pyridine;
N-[7-(4,10-Diazabicyclo[13.3.1]heptadeca-1(17),13,15-trienyl)-1,4-phenylenebis(methylene)]-2-(aminomethyl)pyridine;
N-[4-(11-Fluoro-1,4,7-triazacyclotetradecanyl)-1,4-phenylenebis(methylene)]-2-(aminomethyl)pyridine;
N-[4-(11,11-difluoro-1,4,7-triazacyclotetradecanyl)-1,4-phenylenebis(methylene)]-2-(aminomethyl)pyridine;
N-[4-(1,4,7-triazacyclotetradecan-2-one)-yl))-1,4-phenylenebis(methylene)]-2-(aminomethyl)pyridine;
N-[12-(5-oxa-1,9-diazacyclotetradecanyl)-1,4-phenylenebis(methylene)]-2-(aminomethyl)pyridine;
N-[4-(11-oxa-1,7-diazacyclotetradecanyl)-1,4-phenylenebis(methylene)]-2-(aminomethyl)pyridine;
N-[4-(11-thia-1,7-diazacyclotetradecanyl)-1,4-phenylenebis(methylene)]-2-(aminomethyl)pyridine;
N-[4-(11-sulfoxo-1,7-diazacyclotetradecanyl)-1,4-phenylenebis(methylene)]-2-(aminomethyl)pyridine;
N-[4-(11-sulfono-1,7-diazacyclotetradecanyl)-1,4-phenylenebis(methylene)]-2-(aminomethyl)pyridine;
N-[4-(1,4,7-triazacyclotetradecan-3-one)-yl))-1,4-phenylenebis(methylene)]-2-(aminomethyl)pyridine;
N-(2-pyridinylmethyl)-N′-(6,7,8,9-tetrahydro-5H-cyclohepta[19]pyridin-9-yl)-1,4-benzenedimethanamine;
N-(2-pyridinylmethyl)-N′-(5,6,7,8-tetrahydro-8-quinolinyl)-1,4-benzenedimethanamine;
N-(2-pyridinylmethyl)-N′-(6,7-dihydro-5H-cyclopenta[b]pyridin-7-yl)-1,4-benzenedimethanamine;
N-(2-pyridinylmethyl)-N′-(1,2,3,4-tetrahydro-1-naphthalenyl)-1,4-benzenedimethanamine;
N-(2-pyridinylmethyl)-N′-(1-naphthalenyl)-1,4-benzenedimethanamine;
N-(2-pyridinylmethyl)-N′-(8-quinolinyl)-1,4-benzenedimethanamine;
N-(2-pyridinylmethyl)-N′-[2-[(2-pyridinylmethyl)amino]ethyl]-N′-(1-methyl-1,2,3,4-tetrahydro-8-quinolinyl)-1,4-benzene dimethanamine;
N-(2-pyridinylmethyl)-N′-[2-[(1H-imidazol-2-ylmethyl)amino]ethyl]-N′-(1-methyl-1,2,3,4-tetrahydro-8-quinolinyl)-1,4-benzene dimethanamine;
N-(2-pyridinylmethyl)-N′-(1,2,3,4-tetrahydro-8-quinolinyl)-1,4-benzenedimethanamine;
N-(2-pyridinylmethyl)-N′-[2-[(1H-imidazol-2-ylmethyl)amino]ethyl]-N′-(1,2,3,4-tetrahydro-1-naphthalenyl)-1,4-benzene dimethanamine;
N-(2-pyridinylmethyl)-N′-(2-phenyl-5,6,7,8-tetrahydro-8-quinolinyl)-1,4-benzenedimethanamine;
N,N′-bis(2-pyridinylmethyl)-N′-(2-phenyl-5,6,7,8-tetrahydro-8-quinolinyl)-1,4-benzenedimethanamine;
N-(2-pyridinylmethyl)-N′-(5,6,7,8-tetrahydro-5-quinolinyl)-1,4-benzenedimethanamine;
N-(2-pyridinylmethyl)-N′-(1H-imidazol-2-ylmethyl)-N′-(5,6,7,8-tetrahydro-5-quinolinyl)-1,4-benzenedimethanamine;
N-(2-pyridinylmethyl)-N′-(1H-imidazol-2-ylmethyl)-N′-(5,6,7,8-tetrahydro-8-quinolinyl)-1,4-benzenedimethanamine;
N-(2-pyridinylmethyl)-N′-[(2-amino-3-phenyl)propyl]-N′-(5,6,7,8-tetrahydro-8-quinolinyl)-1,4-benzenedimethanamine;
N-(2-pyridinylmethyl)-N′-(1H-imidazol-4-ylmethyl)-N′-(5,6,7,8-tetrahydro-8-quinolinyl)-1,4-benzenedimethanamine;
N-(2-pyridinylmethyl)-N′-(2-quinolinylmethyl)-N′-(5,6,7,8-tetrahydro-8-quinolinyl)-1,4-benzenedimethanamine;
N-(2-pyridinylmethyl)-N′-(2-(2-naphthoyl)aminoethyl)-N′-(5,6,7,8-tetrahydro-8-quinolinyl)-1,4-benzenedimethanamine;
N-(2-pyridinylmethyl)-N′-[(S)-(2-acetylamino-3-phenyl)propyl]-N′-(5,6,7,8-tetrahydro-8-quinolinyl)-1,4-benzenedimethanamine;
N-(2-pyridinylmethyl)-N′-[(S)-(2-acetylamino-3-phenyl)propyl]-N′-(5,6,7,8-tetrahydro-8-quinolinyl)-1,4-benzenedimethanamine;
N-(2-pyridinylmethyl)-N′-[3-((2-naphthalenylmethyl)amino)propyl]-N′-(5,6,7,8-tetrahydro-8-quinolinyl)-1,4-benzenedimethanamine;
N-(2-pyridinylmethyl)-N′-[2-(S)-pyrollidinylmethyl]-N′-(5,6,7,8-tetrahydro-8-quinolinyl)-1,4-benzenedimethanamine;
N-(2-pyridinylmethyl)-N′-[2-(R)-pyrollidinylmethyl]-N′-(5,6,7,8-tetrahydro-8-quinolinyl)-1,4-benzenedimethanamine;
N-(2-pyridinylmethyl)-N′-[3-pyrazolylmethyl]-N′-(5,6,7,8-tetrahydro-8-quinolinyl)-1,4-benzenedimethanamine;
N-(2-pyridinylmethyl)-N′-[2-pyrrolylmethyl]-N′-(5,6,7,8-tetrahydro-8-quinolinyl)-1,4-benzenedimethanamine;
N-(2-pyridinylmethyl)-N′-[2-thiopheneylmethyl]-N′-(5,6,7,8-tetrahydro-8-quinolinyl)-1,4-benzenedimethanamine
N-(2-pyridinylmethyl)-N′-[2-thiazolylmethyl]-N′-(5,6,7,8-tetrahydro-8-quinolinyl)-1,4-benzenedimethanamine;
N-(2-pyridinylmethyl)-N′-[2-furanylmethyl]-N′-(5,6,7,8-tetrahydro-8-quinolinyl)-1,4-benzenedimethanamine;
N-(2-pyridinylmethyl)-N′-[2-[(phenylmethyl)amino]ethyl]-N′-(5,6,7,8-tetrahydro-8-quinolinyl)-1,4-benzenedimethanamine;
N-(2-pyridinylmethyl)-N′-(2-aminoethyl)-N′-(5,6,7,8-tetrahydro-8-quinolinyl)-1,4-benzenedimethanamine;
N-(2-pyridinylmethyl)-N′-3-pyrrolidinyl-N′-(5,6,7,8-tetrahydro-8-quinolinyl)-1,4-benzenedimethanamine
N-(2-pyridinylmethyl)-N′-4-piperidinyl-N′-(5,6,7,8-tetrahydro-8-quinolinyl)-1,4-benzenedimethanamine;
N-(2-pyridinylmethyl)-N′-[2-[(phenyl)amino]ethyl]-N′-(5,6,7,8-tetrahydro-8-quinolinyl)-1,4-benzenedimethanamine;
N-(2-pyridinylmethyl)-N′-(7-methoxy-1,2,3,4-tetrahydro-2-naphthalenyl)-1,4-benzenedimethanamine;
N-(2-pyridinylmethyl)-N′-(6-methoxy-1,2,3,4-tetrahydro-2-naphthalenyl)-1,4-benzenedimethanamine;
N-(2-pyridinylmethyl)-N′-(1-methyl-1,2,3,4-tetrahydro-2-naphthalenyl)-1,4-benzenedimethanamine;
N-(2-pyridinylmethyl)-N′-(7-methoxy-3,4-dihydronaphthalenyl)-1-(aminomethyl)-4-benzamide;
N-(2-pyridinylmethyl)-N′-(6-methoxy-3,4-dihydronaphthalenyl)-1-(aminomethyl)-4-benzamide;
N-(2-pyridinylmethyl)-N′-(1H-imidazol-2-ylmethyl)-N′-(7-methoxy-1,2,3,4-tetrahydro-2-naphthalenyl)-1,4-benzenedimethanamine;
N-(2-pyridinylmethyl)-N′-(8-hydroxy-1,2,3,4-tetrahydro-2-naphthalenyl)-1,4-benzenedimethanamine;
N-(2-pyridinylmethyl)-N′-(1H-imidazol-2-ylmethyl)-N′-(8-hydroxy-1,2,3,4-tetrahydro-2-naphthalenyl)-1,4-benzenedimethanamine;
N-(2-pyridinylmethyl)-N′-(8-Fluoro-1,2,3,4-tetrahydro-2-naphthalenyl)-1,4-benzenedimethanamine;
N-(2-pyridinylmethyl)-N′-(1H-imidazol-2-ylmethyl)-N′-(8-Fluoro-1,2,3,4-tetrahydro-2-naphthalenyl)-1,4-benzenedimethanamine;
N-(2-pyridinylmethyl)-N′-(5,6,7,8-tetrahydro-7-quinolinyl)-1,4-benzenedimethanamine;
N-(2-pyridinylmethyl)-N′-(1H-imidazol-2-ylmethyl)-N′-(5,6,7,8-tetrahydro-7-quinolinyl)-1,4-benzenedimethanamine;
N-(2-pyridinylmethyl)-N′-[2-[(2-naphthalenylmethyl)amino]ethyl]-N′-(5,6,7,8-tetrahydro-8-quinolinyl)-1,4-benzenedimethanamine;
N-(2-pyridinylmethyl)-N′-[2-(isobutylamino)ethyl]-N′-(5,6,7,8-tetrahydro-8-quinolinyl)-1,4-benzenedimethanamine;
N-(2-pyridinylmethyl)-N′-[2-[(2-pyridinylmethyl)amino]ethyl]-N′-(5,6,7,8-tetrahydro-8-quinolinyl)-1,4-benzenedimethanamine;
N-(2-pyridinylmethyl)-N′-[2-[(2-furanylmethyl)amino]ethyl]-N′-(5,6,7,8-tetrahydro-8-quinolinyl)-1,4-benzenedimethanamine;
N-(2-pyridinylmethyl)-N′-(2-guanidinoethyl)-N′-(5,6,7,8-tetrahydro-8-quinolinyl)-1,4-benzenedimethanamine;
N-(2-pyridinylmethyl)-N′-[2-[bis-[(2-methoxy)phenylmethyl]amino]ethyl]-N′-(5,6,7,8-tetrahydro-8-quinolinyl)-1,4-benzene dimethanamine;
N-(2-pyridinylmethyl)-N′-[2-[(1H-imidazol-4-ylmethyl)amino]ethyl]-N′-(5,6,7,8-tetrahydro-8-quinolinyl)-1,4-benzene dimethanamine;
N-(2-pyridinylmethyl)-N′-[2-[(1H-imidazol-2-ylmethyl)amino]ethyl]-N′-(5,6,7,8-tetrahydro-8-quinolinyl)-1,4-benzenedimethanamine;
N-(2-pyridinylmethyl)-N′-[2-(phenylureido)ethyl]-N′-(5,6,7,8-tetrahydro-8-quinolinyl)-1,4-benzenedimethanamine;
N-(2-pyridinylmethyl)-N′-[[N″-(n-butyl)carboxamido]methyl]-N′-(5,6,7,8-tetrahydro-8-quinolinyl)-1,4-benzenedimethanamine;
N-(2-pyridinylmethyl)-N′-(carboxamidomethyl)-N′-(5,6,7,8-tetrahydro-8-quinolinyl)-1,4-benzenedimethanamine;
N-(2-pyridinylmethyl)-N′-[(N″-phenyl)carboxamidomethyl]-N′-(5,6,7,8-tetrahydro-8-quinolinyl)-1,4-benzenedimethanamine;
N-(2-pyridinylmethyl)-N′-(carboxymethyl)-N′-(5,6,7,8-tetrahydro-8-quinolinyl)-1,4-benzenedimethanamine;
N-(2-pyridinylmethyl)-N′-(phenylmethyl)-N′-(5,6,7,8-tetrahydro-8-quinolinyl)-1,4-benzenedimethanamine;
N-(2-pyridinylmethyl)-N′-(1H-benzimidazol-2-ylmethyl)-N′-(5,6,7,8-tetrahydro-8-quinolinyl)-1,4-benzenedimethanamine;
N-(2-pyridinylmethyl)-N′-(5,6-dimethyl-1H-benzimidazol-2-ylmethyl)-N′-(5,6,7,8-tetrahydro-8-quinolinyl)-1,4-benzenedimethanamine;
N-(2-pyridinylmethyl)-N′-(5-nitro-1H-benzimidazol-2-ylmethyl)-N′-(5,6,7,8-tetrahydro-8-quinolinyl)-1,4-benzenedimethanamine;
N-(2-pyridinylmethyl)-N′-[(1H)-5-azabenzimidazol-2-ylmethyl]-N′-(5,6,7,8-tetrahydro-8-quinolinyl)-1,4-benzenedimethanamine;
N-(2-pyridinylmethyl)-N-(4-phenyl-1H-imidazol-2-ylmethyl)-N′-(5,6,7,8-tetrahydro-8-quinolinyl)-1,4-benzenedimethanamine;
N-(2-pyridinylmethyl)-N′-[2-(2-pyridinyl)ethyl]-N′-(5,6,7,8-tetrahydro-8-quinolinyl)-1,4-benzenedimethanamine;
N-(2-pyridinylmethyl)-N′-(2-benzoxazolyl)-N′-(5,6,7,8-tetrahydro-8-quinolinyl)-1,4-benzenedimethanamine;
N-(2-pyridinylmethyl)-N′-(trans-2-aminocyclohexyl)-N′-(5,6,7,8-tetrahydro-8-quinolinyl)-1,4-benzenedimethanamine;
N-(2-pyridinylmethyl)-N′-(2-phenylethyl)-N′-(5,6,7,8-tetrahydro-8-quinolinyl)-1,4-benzenedimethanamine;
N-(2-pyridinylmethyl)-N′-(3-phenylpropyl)-N′-(5,6,7,8-tetrahydro-8-quinolinyl)-1,4-benzenedimethanamine;
N-(2-pyridinylmethyl)-N′-(trans-2-aminocyclopentyl)-N′-(5,6,7,8-tetrahydro-8-quinolinyl)-1,4-benzenedimethanamine;
N-[[4-[[(2-pyridinylmethyl)amino]methyl]phenyl]methyl]-N-(5,6,7,8-tetrahydro-8-quinolinyl)-glycinamide;
N-[[4-[[(2-pyridinylmethyl)amino]methyl]phenyl]methyl]-N-(5,6,7,8-tetrahydro-8-quinolinyl)-(L)-alaninamide;
N-[[4-[[(2-pyridinylmethyl)amino]methyl]phenyl]methyl]-N-(5,6,7,8-tetrahydro-8-quinolinyl)-(L)-aspartamide;
N-[[4-[[(2-pyridinylmethyl)amino]methyl]phenyl]methyl]-N-(5,6,7,8-tetrahydro-8-quinolinyl)-pyrazinamide;
N-[[4-[[(2-pyridinylmethyl)amino]methyl]phenyl]methyl]-N-(5,6,7,8-tetrahydro-8-quinolinyl)-(L)-prolinamide;
N-[[4-[[(2-pyridinylmethyl)amino]methyl]phenyl]methyl]-N-(5,6,7,8-tetrahydro-8-quinolinyl)-(L)-lysinamide;
N-[[4-[[(2-pyridinylmethyl)amino]methyl]phenyl]methyl]-N-(5,6,7,8-tetrahydro-8-quinolinyl)-benzamide;
N-[[4-[[(2-pyridinylmethyl)amino]methyl]phenyl]methyl]-N-(5,6,7,8-tetrahydro-8-quinolinyl)-picolinamide;
N′-Benzyl-N-[[4-[[(2-pyridinylmethyl)amino]methyl]phenyl]methyl]-N-(5,6,7,8-tetrahydro-8-quinolinyl)-urea;
N′-phenyl-N-[[4-[[(2-pyridinylmethyl)amino]methyl]phenyl]methyl]-N-(5,6,7,8-tetrahydro-8-quinolinyl)-urea;
N-(6,7,8,9-tetrahydro-5H-cyclohepta[bacteriapyridin-9-yl)-4-[[(2-pyridinylmethyl)amino]methyl]benzamide;
N-(5,6,7,8-tetrahydro-8-quinolinyl)-4-[[(2-pyridinylmethyl)amino]methyl]benzamide;
N,N′-bis(2-pyridinylmethyl)-N′-(5,6,7,8-tetrahydro-8-quinolinyl)-1,4-benzenedimethanamine;
N,N′-bis(2-pyridinylmethyl)-N′-(6,7,8,9-tetrahydro-5H-cyclohepta[bacteriapyridin-9-yl)-1,4-benzenedimethanamine;
N,N′-bis(2-pyridinylmethyl)-N′-(6,7-dihydro-5H-cyclopenta[bacteriapyridin-7-yl)-1,4-benzenedimethanamine;
N,N′-bis(2-pyridinylmethyl)-N′-(1,2,3,4-tetrahydro-1-naphthalenyl)-1,4-benzenedimethanamine;
N,N′-bis(2-pyridinylmethyl)-N′-[(5,6,7,8-tetrahydro-8-quinolinyl)methyl]-1,4-benzenedimethanamine;
N,N′-bis(2-pyridinylmethyl)-N′[(6,7-dihydro-5H-cyclopenta[bacteriapyridin-7-yl)methyl]-1,4-benzenedimethanamine;
N-(2-pyridinylmethyl)-N-(2-methoxyethyl)-N′-(5,6,7,8-tetrahydro-8-quinolinyl)-1,4-benzenedimethanamine;
N-(2-pyridinylmethyl)-N-[2-(4-methoxyphenyl)ethyl]-N′-(5,6,7,8-tetrahydro-8-quinolinyl)-1,4-benzenedimethanamine;
N,N′-bis(2-pyridinylmethyl)-1,4-(5,6,7,8-tetrahydro-8-quinolinyl)benzenedimethanamine;
N-[(2,3-dimethoxyphenyl)methyl]-N′-(2-pyridinylmethyl)-N-(5,6,7,8-tetrahydro-8-quinolinyl)-1,4-benzenedimethanamine;
N,N′-bis(2-pyridinylmethyl)-N-[1-(N″-phenyl-N″-methylureido)-4-piperidinyl]-1,3-benzenedimethanamine;
N,N′-bis(2-pyridinylmethyl)-N—[N″-p-toluenesulfonylphenylalanyl)-4-piperidinyl]-1,3-benzenedimethanamine;
N,N′-bis(2-pyridinylmethyl)-N-[1-[3-(2-chlorophenyl)-5-methyl-isoxazol-4-oyl]-4-piperidinyl]-1,3-benzenedimethanamine;
N-[(2-hydroxyphenyl)methyl]-N′-(2-pyridinylmethyl)-N-(6,7,8,9-tetrahydro-5H-cyclohepta[bacteriapyridin-9-yl)-1,4-benzenedimethanamine;
N-[(4-cyanophenyl)methyl]-N′-(2-pyridinylmethyl)-N-(6,7,8,9-tetrahydro-5H-cyclohepta[bacteriapyridin-9-yl)-1,4-benzenedimethanamine;
N-[(4-cyanophenyl)methyl]-N′-(2-pyridinylmethyl)-N-(5,6,7,8-tetrahydro-8-quinolinyl)-1,4-benzenedimethanamine;
N-[(4-acetamidophenyl)methyl]-N′-(2-pyridinylmethyl)-N-(5,6,7,8-tetrahydro-8-quinolinyl)-1,4-benzenedimethanamine;
N-[(4-phenoxyphenyl)methyl]-N′-(2-pyridinylmethyl)-N-(6,7,8,9-tetrahydro-5H-cyclohepta[bacteriapyridin-9-yl)-1,4-benzenedimethanamine;
N-[(1-methyl-2-carboxamido)ethyl]-N,N′-bis(2-pyridinylmethyl)-1,3-benzenedimethanamine;
N-[(4-benzyloxyphenyl)methyl]-N′-(2-pyridinylmethyl)-N-(6,7,8,9-tetrahydro-5H-cyclohepta[bacteriapyridin-9-yl)-1,4-benzenedimethanamine;
N-[(thiophene-2-yl)methyl]-N′-(2-pyridinylmethyl)-N-(6,7,8,9-tetrahydro-5H-cyclohepta[bacteriapyridin-9-yl)-1,4-benzenedimethanamine;
N-[1-(benzyl)-3-pyrrolidinyl]-N,N′-bis(2-pyridinylmethyl)-1,3-benzenedimethanamine;
N-[[1-methyl-3-(pyrazol-3-yl)]propyl]-N,N′-bis(2-pyridinylmethyl)-1,3-benzenedimethanamine;
N-[1-(phenyl)ethyl]-N,N′-bis(2-pyridinylmethyl)-1,3-benzenedimethanamine;
N-[(3,4-methylenedioxyphenyl)methyl]-N′-(2-pyridinylmethyl)-N-(6,7,8,9-tetrahydro-5H-cyclohepta[b]pyridin-9-yl)-1,4-benzenedimethanamine;
N-[1-benzyl-3-carboxymethyl-4-piperidinyl]-N,N′-bis(2-pyridinylmethyl)-1,3-benzenedimethanamine;
N-[(3,4-methylenedioxyphenyl)methyl]-N′-(2-pyridinylmethyl)-N-(5,6,7,8-tetrahydro-8-quinolinyl)-1,4-benzenedimethanamine;
N-(3-pyridinylmethyl)-N′-(2-pyridinylmethyl)-N-(6,7,8,9-tetrahydro-5H-cyclohepta[b]pyridin-9-yl)-1,4-benzenedimethanamine;
N-[[1-methyl-2-(2-tolyl)carboxamido]ethyl]-N,N′-bis(2-pyridinylmethyl)-1,3-benzenedimethanamine;
N-[(1,5-dimethyl-2-phenyl-3-pyrazolinone-4-yl)methyl]-N′-(2-pyridinylmethyl)-N-(5,6,7,8-tetrahydro-8-quinolinyl)-1,4-benzenedimethanamine;
N-[(4-propoxyphenyl)methyl]-N′-(2-pyridinylmethyl)-N-(6,7,8,9-tetrahydro-5H-cyclohepta[b]pyridin-9-yl)-1,4-benzenedimethanamine;
N-(1-phenyl-3,5-dimethylpyrazolin-4-ylmethyl)-N′-(2-pyridinylmethyl)-N-(5,6,7,8-tetrahydro-8-quinolinyl)-1,4-benzenedimethanamine;
N-[1H-imidazol-4-ylmethyl]-N,N′-bis(2-pyridinylmethyl)-1,3-benzenedimethanamine;
N-[(3-methoxy-4,5-methylenedioxyphenyl)methyl]-N′-(2-pyridinylmethyl)-N-(6,7,8,9-tetrahydro-5H-cyclohepta[b]pyridin-9-yl)-1,4-benzenedimethanamine;
N-[(3-cyanophenyl)methyl]-N′-(2-pyridinylmethyl)-N-(6,7,8,9-tetrahydro-5H-cyclohepta[b]pyridin-9-yl)-1,4-benzenedimethanamine;
N-[(3-cyanophenyl)methyl]-N′-(2-pyridinylmethyl)-N-(5,6,7,8-tetrahydro-8-quinolinyl)-1,4-benzenedimethanamine;
N-(5-ethylthiophene-2-ylmethyl)-N′-(2-pyridinylmethyl)-N-(6,7,8,9-tetrahydro-5H-cyclohepta[b]pyridin-9-yl)-1,4-benzenedimethanamine;
N-(5-ethylthiophene-2-ylmethyl)-N′-(2-pyridinylmethyl)-N-(5,6,7,8-tetrahydro-8-quinolinyl)-1,4-benzenedimethanamine;
N-[(2,6-difluorophenyl)methyl]-N′-(2-pyridinylmethyl)-N-(6,7,8,9-tetrahydro-5H-cyclohepta[b]pyridin-9-yl)-1,4-benzenedimethanamine;
N-[(2,6-difluorophenyl)methyl]-N′-(2-pyridinylmethyl)-N-(5,6,7,8-tetrahydro-8-quinolinyl)-1,4-benzenedimethanamine;
N-[(2-difluoromethoxyphenyl)methyl]-N′-(2-pyridinylmethyl)-N-(6,7,8,9-tetrahydro-5H-cyclohepta[b]pyridin-9-yl)-1,4-benzenedimethanamine;
N-(2-difluoromethoxyphenylmethyl)-N′-(2-pyridinylmethyl)-N-(5,6,7,8-tetrahydro-8-quinolinyl)-1,4-benzenedimethanamine;
N-(1,4-benzodioxan-6-ylmethyl)-N′-(2-pyridinylmethyl)-N-(6,7,8,9-tetrahydro-5H-cyclohepta[b]pyridin-9-yl)-1,4-benzenedimethanamine;
N,N′-bis(2-pyridinylmethyl)-N-[1-(N″-phenyl-N″-methylureido)-4-piperidinyl]-1,4-benzenedimethanamine;
N,N′-bis(2-pyridinylmethyl)-N—[N″-p-toluenesulfonylphenylalanyl)-4-piperidinyl]-1,4-benzenedimethanamine;
N-[1-(3-pyridinecarboxamido)-4-piperidinyl]-N,N′-bis(2-pyridinylmethyl)-1,4-benzenedimethanamine;
N-[1-(cyclopropylcarboxamido)-4-piperidinyl]-N,N′-bis(2-pyridinylmethyl)-1,4-benzenedimethanamine;
N-[1-(1-phenylcyclopropylcarboxamido)-4-piperidinyl]-N,N′-bis(2-pyridinylmethyl)-1,4-benzenedimethanamine;
N-(1,4-benzodioxan-6-ylmethyl)-N′-(2-pyridinylmethyl)-N-(5,6,7,8-tetrahydro-8-quinolinyl)-1,4-benzenedimethanamine;
N-[1-[3-(2-chlorophenyl)-5-methyl-isoxazol-4-carboxamido]-4-piperidinyl]-N,N′-bis(2-pyridinylmethyl)-1,4-benzenedimethanamine;
N-[1-(2-thiomethylpyridine-3-carboxamido)-4-piperidinyl]-N,N′-bis(2-pyridinylmethyl)-1,4-benzenedimethanamine;
N-[(2,4-difluorophenyl)methyl]-N′-(2-pyridinylmethyl)-N-(5,6,7,8-tetrahydro-8-quinolinyl)-1,4-benzenedimethanamine;
N-(1-methylpyrrol-2-ylmethyl)-N′-(2-pyridinylmethyl)-N-(5,6,7,8-tetrahydro-8-quinolinyl)-1,4-benzenedimethanamine;
N-[(2-hydroxyphenyl)methyl]-N′-(2-pyridinylmethyl)-N-(5,6,7,8-tetrahydro-8-quinolinyl)-1,4-benzenedimethanamine;
N-[(3-methoxy-4,5-methylenedioxyphenyl)methyl]-N′-(2-pyridinylmethyl)-N-(5,6,7,8-tetrahydro-8-quinolinyl)-1,4-benzenedimethanamine;
N-(3-pyridinylmethyl)-N′-(2-pyridinylmethyl)-N-(5,6,7,8-tetrahydro-8-quinolinyl)-1,4-benzenedimethanamine;
N-[2-(N″-morpholinomethyl)-1-cyclopentyl]-N,N′-bis(2-pyridinylmethyl)-1,4-benzenedimethanamine;
N-[(1-methyl-3-piperidinyl)propyl]-N,N′-bis(2-pyridinylmethyl)-1,4-benzenedimethanamine;
N-(1-methylbenzimidazol-2-ylmethyl)-N′-(2-pyridinylmethyl)-N-(5,6,7,8-tetrahydro-8-quinolinyl)-1,4-benzenedimethanamine;
N-[1-(benzyl)-3-pyrrolidinyl]-N,N′-bis(2-pyridinylmethyl)-1,4-benzenedimethanamine;
N-[[(1-phenyl-3-(N″-morpholino)]propyl]-N,N′-bis(2-pyridinylmethyl)-1,4-benzenedimethanamine;
N-[1-(iso-propyl)-4-piperidinyl]-N,N′-bis(2-pyridinylmethyl)-1,4-benzenedimethanamine;
N-[1-(ethoxycarbonyl)-4-piperidinyl]-N′-(2-pyridinylmethyl)-N-(5,6,7,8-tetrahydro-8-quinolinyl)-1,4-benzenedimethanamine;
N-[(1-methyl-3-pyrazolyl)propyl]-N′-(2-pyridinylmethyl)-N-(5,6,7,8-tetrahydro-8-quinolinyl)-1,4-benzenedimethanamine;
N-[1-methyl-2-(N″,N″-diethylcarboxamido)ethyl]-N,N′-bis(2-pyridinylmethyl)-1,4-benzenedimethanamine;
N-[(1-methyl-2-phenylsulfonyl)ethyl]-N′-(2-pyridinylmethyl)-N-(5,6,7,8-tetrahydro-8-quinolinyl)-1,4-benzenedimethanamine;
N-[(2-chloro-4,5-methylenedioxyphenyl)methyl]-N′-(2-pyridinylmethyl)-N-(5,6,7,8-tetrahydro-8-quinolinyl)-1,4-benzenedimethanamine;
N-[1-methyl-2-[N″-(4-chlorophenyl)carboxamido]ethyl]-N′-(2-pyridinylmethyl)-N-(5,6,7,8-tetrahydro-8-quinolinyl)-1,4-benzenedimethanamine;
N-(1-acetoxyindole-3-ylmethyl)-N′-(2-pyridinylmethyl)-N-(6,7,8,9-tetrahydro-5H-cyclohepta[b]pyridin-9-yl)-1,4-benzenedimethanamine;
N-[(3-benzyloxy-4-methoxyphenyl)methyl]-N′-(2-pyridinylmethyl)-N-(6,7,8,9-tetrahydro-5H-cyclohepta[b]pyridin-9-yl)-1,4-benzenedimethanamine;
N-(3-quinolylmethyl)-N′-(2-pyridinylmethyl)-N-(5,6,7,8-tetrahydro-8-quinolinyl)-1,4-benzenedimethanamine;
N-[(8-hydroxy)-2-quinolylmethyl]-N′-(2-pyridinylmethyl)-N-(6,7,8,9-tetrahydro-5H-cyclohepta[b]pyridin-9-yl)-1,4-benzenedimethanamine;
N-(2-quinolylmethyl)-N′-(2-pyridinylmethyl)-N-(6,7,8,9-tetrahydro-5H-cyclohepta[b]pyridin-9-yl)-1,4-benzenedimethanamine;
N-[(4-acetamidophenyl)methyl]-N′-(2-pyridinylmethyl)-N-(6,7,8,9-tetrahydro-5H-cyclohepta[b]pyridin-9-yl)-1,4-benzenedimethanamine;
N-[1H-imidazol-2-ylmethyl]-N,N′-bis(2-pyridinylmethyl)-1,4-benzenedimethanamine;
N-(3-quinolylmethyl)-N′-(2-pyridinylmethyl)-N-(6,7,8,9-tetrahydro-5H-cyclohepta[b]pyridin-9-yl)-1,4-benzenedimethanamine;
N-(2-thiazolylmethyl)-N′-(2-pyridinylmethyl)-N-(6,7,8,9-tetrahydro-5H-cyclohepta[b]pyridin-9-yl)-1,4-benzenedimethanamine;
N-(4-pyridinylmethyl)-N′-(2-pyridinylmethyl)-N-(6,7,8,9-tetrahydro-5H-cyclohepta[b]pyridin-9-yl)-1,4-benzenedimethanamine;
N-[(5-benzyloxy)benzo[b]pyrrol-3-ylmethyl]-N,N′-bis(2-pyridinylmethyl)-1,4-benzenedimethanamine;
N-(1-methylpyrazol-2-ylmethyl)-N′-(2-pyridinylmethyl)-N-(6,7,8,9-tetrahydro-5H-cyclohepta[b]pyridin-9-yl)-1,4-benzenedimethanamine;
N-[(4-methyl)-1H-imidazol-5-ylmethyl]-N,N′-bis(2-pyridinylmethyl)-1,4-benzenedimethanamine;
N-[[(4-dimethylamino)-1-napthalenyl]methyl]-N,N′-bis(2-pyridinylmethyl)-1,4-benzenedimethanamine;
N-[1,5-dimethyl-2-phenyl-3-pyrazolinone-4-ylmethyl]-N,N′-bis(2-pyridinylmethyl)-1,4-benzenedimethanamine;
N-[1-[(1-acetyl-2-(R)-prolinyl]-4-piperidinyl]-N-[2-(2-pyridinyl)ethyl]-N′-(2-pyridinylmethyl)-1,3-benzenedimethanamine;
N-[1-[2-acetamidobenzoyl-4-piperidinyl]-4-piperidinyl]-N-[2-(2-pyridinyl)ethyl]-N′-(2-pyridinylmethyl)-1,3-benzenedimethanamine;
N-[(2-cyano-2-phenyl)ethyl]-N′-(2-pyridinylmethyl)-N-(6,7,8,9-tetrahydro-5H-cyclohepta[b]pyridin-9-yl)-1,4-benzenedimethanamine;
N—[(N″-acetyltryptophanyl)-4-piperidinyl]-N-[2-(2-pyridinyl)ethyl]-N′-(2-pyridinylmethyl)-1,3-benzenedimethanamine;
N—[(N″-benzoylvalinyl)-4-piperidinyl]-N-[2-(2-pyridinyl)ethyl]-N′-(2-pyridinylmethyl)-1,3-benzenedimethanamine;
N-[(4-dimethylaminophenyl)methyl]-N′-(2-pyridinylmethyl)-N-(6,7,8,9-tetrahydro-5H-cyclohepta[b]pyridin-9-yl)-1,4-benzenedimethanamine;
N-(4-pyridinylmethyl)-N′-(2-pyridinylmethyl)-N-(5,6,7,8-tetrahydro-8-quinolinyl)-1,4-benzenedimethanamine;
N-(1-methylbenzimidazol-2-ylmethyl)-N′-(2-pyridinylmethyl)-N-(6,7,8,9-tetrahydro-5H-cyclohepta[b]pyridin-9-yl)-1,4-benzenedimethanamine;
N-[1-butyl-4-piperidinyl]-N-[2-(2-pyridinyl)ethyl]-N′-(2-pyridinylmethyl)-1,3-benzenedimethanamine;
N-[1-benzoyl-4-piperidinyl]-N-[2-(2-pyridinyl)ethyl]-N′-(2-pyridinylmethyl)-1,3-benzenedimethanamine;
N-[1-(benzyl)-3-pyrrolidinyl]-N-[2-(2-pyridinyl)ethyl]-N′-(2-pyridinylmethyl)-1,3-benzenedimethanamine;
N-[1-methyl)benzo[b]pyrrol-3-ylmethyl]-N-[2-(2-pyridinyl)ethyl]-N′-(2-pyridinylmethyl)-1,3-benzenedimethanamine;
N-[1H-imidazol-4-ylmethyl]-N-[2-(2-pyridinyl)ethyl]-N′-(2-pyridinylmethyl)-1,3-benzenedimethanamine;
N-[1-(benzyl)-4-piperidinyl]-N-[2-(2-pyridinyl)ethyl]-N′-(2-pyridinylmethyl)-1,4-benzenedimethanamine;
N-[1-methylbenzimidazol-2-ylmethyl]-N-[2-(2-pyridinyl)ethyl]-N′-(2-pyridinylmethyl)-1,4-benzenedimethanamine;
N-[(2-phenyl)benzo[b]pyrrol-3-ylmethyl]-N-[2-(2-pyridinyl)ethyl]-N′-(2-pyridinylmethyl)-1,4-benzenedimethanamine;
N-[(6-methylpyridin-2-yl)methyl]-N′-(2-pyridinylmethyl)-N-(5,6,7,8-tetrahydro-8-quinolinyl)-1,4-benzenedimethanamine;
N-(3-methyl-1H-pyrazol-5-ylmethyl)-N′-(2-pyridinylmethyl)-N-(5,6,7,8-tetrahydro-8-quinolinyl)-1,3-benzenedimethanamine;
N-[(2-methoxyphenyl)methyl]-N′-(2-pyridinylmethyl)-N-(5,6,7,8-tetrahydro-8-quinolinyl)-1,3-benzenedimethanamine;
N-[(2-ethoxyphenyl)methyl]-N′-(2-pyridinylmethyl)-N-(6,7,8,9-tetrahydro-5H-cyclohepta[b]pyridin-9-yl)-1,3-benzenedimethanamine;
N-(benzyloxyethyl)-N′-(2-pyridinylmethyl)-N-(5,6,7,8-tetrahydro-8-quinolinyl)-1,3-benzenedimethanamine;
N-[(2-ethoxy-1-naphthalenyl)methyl]-N′-(2-pyridinylmethyl)-N-(5,6,7,8-tetrahydro-8-quinolinyl)-1,3-benzenedimethanamine;
N-[(6-methylpyridin-2-yl)methyl]-N′-(2-pyridinylmethyl)-N-(5,6,7,8-tetrahydro-8-quinolinyl)-1,3-benzenedimethanamine;
1-[[4-[[(2-pyridinylmethyl)amino]methyl]phenyl]methyl]guanidine;
N-(2-pyridinylmethyl)-N-(8-methyl-8-azabicyclo[3.2.1]octan-3-yl)-1,4-benzenedimethanamine;
1-[[4-[[(2-pyridinylmethyl)amino]methyl]phenyl]methyl]homopiperazine;
1-[[3-[[(2-pyridinylmethyl)amino]methyl]phenyl]methyl]homopiperazine;
trans and cis-1-[[4-[[(2-pyridinylmethyl)amino]methyl]phenyl]methyl]-3,5-piperidinediamine;
N,N′-[1,4-Phenylenebis(methylene)]bis-4-(2-pyrimidyl)piperazine;
1-[[4-[[(2-pyridinylmethyl)amino]methyl]phenyl]methyl]-1-(2-pyridinyl)methylamine;
2-(2-pyridinyl)-5-[[(2-pyridinylmethyl)amino]methyl]-1,2,3,4-tetrahydroisoquinoline;
1-[[4-[[(2-pyridinylmethyl)amino]methyl]phenyl]methyl]-3,4-diaminopyrrolidine;
1-[[4-[[(2-pyridinylmethyl)amino]methyl]phenyl]methyl]-3,4-diacetylaminopyrrolidine;
8-[[4-[[(2-pyridinylmethyl)amino]methyl]phenyl]methyl]-2,5,8-triaza-3-oxabicyclo[4.3.0]nonane;
8-[[4-[[(2-pyridinylmethyl)amino]methyl]phenyl]methyl]-2,5,8-triazabicyclo[4.3.0]nonane;
(4-Aminomethyl-pyridin-3-ylmethyl)-(1H-benzoimidazol-2-ylmethyl)-(5,6,7,8-tetrahydro-quinolin-8-yl)-amine;
(3-Aminomethyl-pyridin-4-ylmethyl)-(1H-benzoimidazol-2-ylmethyl)-(5,6,7,8-tetrahydro-quinolin-8-yl)-amine;
1-(3-Aminomethyl-4-{[(1H-benzoimidazol-2-ylmethyl)-(5,6,7,8-tetrahydro-quinolin-8-yl)-amino]-methyl}-phenyl)-ethanone;
1-(5-Aminomethyl-2-{[(1H-benzoimidazol-2-ylmethyl)-(5,6,7,8-tetrahydro-quinolin-8-yl)-amino]-methyl}-phenyl)-ethanone;
3-Aminomethyl-4-{[(1H-benzoimidazol-2-ylmethyl)-(5,6,7,8-tetrahydro-quinolin-8-yl)-amino]-methyl}-benzenesulfonamide;
5-Aminomethyl-2-{[(1H-benzoimidazol-2-ylmethyl)-(5,6,7,8-tetrahydro-quinolin-8-yl)-amino]-methyl}-benzenesulfonamide;
N-(3-Aminomethyl-4-{[(1H-benzoimidazol-2-ylmethyl)-(5,6,7,8-tetrahydro-quinolin-8-yl)-amino]-methyl}-benzyl)-hydroxylamine;
N-(5-Aminomethyl-2-{[(1H-benzoimidazol-2-ylmethyl)-(5,6,7,8-tetrahydro-quinolin-8-yl)-amino]-methyl}-benzyl)-hydroxylamine;
N-(3-Aminomethyl-4-{[(1H-benzoimidazol-2-ylmethyl)-(5,6,7,8-tetrahydro-quinolin-8-yl)-amino]-methyl}-benzyl)-O-methyl-hydroxylamine;
N-(5-Aminomethyl-2-{[(1H-benzoimidazol-2-ylmethyl)-(5,6,7,8-tetrahydro-quinolin-8-yl)-amino]-methyl}-benzyl)-O-methyl-hydroxylamine;
(4-Aminomethyl-2-methoxymethyl-benzyl)-(1H-benzoimidazol-2-ylmethyl)-(5,6,7,8-tetrahydro-quinolin-8-yl)-amine;
(2-Aminomethyl-4-methoxymethyl-benzyl)-(1H-benzoimidazol-2-ylmethyl)-(5,6,7,8-tetrahydro-quinolin-8-yl)-amine;
N-(2-{[(1H-Benzoimidazol-2-ylmethyl)-(5,6,7,8-tetrahydro-quinolin-8-yl)-amino]-methyl}-benzyl)-formamide;
N-(4-{[(1H-Benzoimidazol-2-ylmethyl)-(5,6,7,8-tetrahydro-quinolin-8-yl)-amino]-methyl}-benzyl)-formamide;
N-(2-{[(1H-Benzoimidazol-2-ylmethyl)-(5,6,7,8-tetrahydro-quinolin-8-yl)-amino]-methyl}-benzyl)-hydroxylamine;
(1H-Benzoimidazol-2-ylmethyl)-(2,6-bis-aminomethyl-benzyl)-(5,6,7,8-tetrahydro-quinolin-8-yl)-amine;
(3-Aminomethyl-2-{[(1H-benzoimidazol-2-ylmethyl)-(5,6,7,8-tetrahydro-quinolin-8-yl)-amino]-methyl}-phenyl)-methanol;
(2-Aminomethyl-6-methoxymethyl-benzyl)-(1H-benzoimidazol-2-ylmethyl)-(5,6,7,8-tetrahydro-quinolin-8-yl)-amine;
N-(3-Aminomethyl-2-{[(1H-benzoimidazol-2-ylmethyl)-(5,6,7,8-tetrahydro-quinolin-8-yl)-amino]-methyl}-benzyl)-hydroxylamine;
N-(3-Aminomethyl-2-{[(1H-benzoimidazol-2-ylmethyl)-(5,6,7,8-tetrahydro-quinolin-8-yl)-amino]-methyl}-benzyl)-O-methyl-hydroxylamine;
[2-Aminomethyl-4-(1H-imidazol-2-yl)-benzyl]-(1H-benzoimidazol-2-ylmethyl)-(5,6,7,8-tetrahydro-quinolin-8-yl)-amine;
[2-Aminomethyl-4-(1-methyl-1H-imidazol-2-yl)-benzyl]-(1H-benzoimidazol-2-ylmethyl)-(5,6,7,8-tetrahydro-quinolin-8-yl)-amine;
[2-Aminomethyl-4-(2H-pyrazol-3-yl)-benzyl]-(1H-benzoimidazol-2-ylmethyl)-(5,6,7,8-tetrahydro-quinolin-8-yl)-amine;
[2-Aminomethyl-4-(1-methyl-1H-pyrazol-3-yl)-benzyl]-(1H-benzoimidazol-2-ylmethyl)-(5,6,7,8-tetrahydro-quinolin-8-yl)-amine;
[2-Aminomethyl-4-(1H-[1,2,4]triazol-3-yl)-benzyl]-(1H-benzoimidazol-2-ylmethyl)-(5,6,7,8-tetrahydro-quinolin-8-yl)-amine;
[2-Aminomethyl-4-(1-methyl-1H-[1,2,4]triazol-3-yl)-benzyl]-(1H-benzoimidazol-2-ylmethyl)-(5,6,7,8-tetrahydro-quinolin-8-yl)-amine;
(2-Aminomethyl-4-oxazol-2-yl-benzyl)-(1H-benzoimidazol-2-ylmethyl)-(5,6,7,8-tetrahydro-quinolin-8-yl)-amine;
(2-Aminomethyl-4-furan-2-yl-benzyl)-(1H-benzoimidazol-2-ylmethyl)-(5,6,7,8-tetrahydro-quinolin-8-yl)-amine;
[2-Aminomethyl-4-(tetrahydro-furan-2-yl)-benzyl]-(1H-benzoimidazol-2-ylmethyl)-(5,6,7,8-tetrahydro-quinolin-8-yl)-amine;
(2-Aminomethyl-4-thiazol-2-yl-benzyl)-(1H-benzoimidazol-2-ylmethyl)-(5,6,7,8-tetrahydro-quinolin-8-yl)-amine;
[2-Aminomethyl-4-(1H-tetrazol-5-yl)-benzyl]-(1H-benzoimidazol-2-ylmethyl)-(5,6,7,8-tetrahydro-quinolin-8-yl)-amine;
[2-Aminomethyl-4-(2-methyl-2H-tetrazol-5-yl)-benzyl]-(1H-benzoimidazol-2-ylmethyl)-(5,6,7,8-tetrahydro-quinolin-8-yl)-amine;
(2-Aminomethyl-4-pyridin-2-yl-benzyl)-(1H-benzoimidazol-2-ylmethyl)-(5,6,7,8-tetrahydro-quinolin-8-yl)-amine;
(2-Aminomethyl-4-piperidin-2-yl-benzyl)-(1H-benzoimidazol-2-ylmethyl)-(5,6,7,8-tetrahydro-quinolin-8-yl)-amine;
(4-Aminomethyl-3-{[(1H-benzoimidazol-2-ylmethyl)-(5,6,7,8-tetrahydro-quinolin-8-yl)-amino]-methyl}-phenyl)-methanol;
(2-Aminomethyl-5-methoxymethyl-benzyl)-(1H-benzoimidazol-2-ylmethyl)-(5,6,7,8-tetrahydro-quinolin-8-yl)-amine;
(4-Aminomethyl-5-{[(1H-benzoimidazol-2-ylmethyl)-(5,6,7,8-tetrahydro-quinolin-8-yl)-amino]-methyl}-pyridin-2-yl)-methanol;
(4-Aminomethyl-6-methoxymethyl-pyridin-3-ylmethyl)-(1H-benzoimidazol-2-ylmethyl)-(5,6,7,8-tetrahydro-quinolin-8-yl)-amine;
(1H-Benzoimidazol-2-ylmethyl)-(4,6-bis-aminomethyl-pyridin-3-ylmethyl)-(5,6,7,8-tetrahydro-quinolin-8-yl)-amine;
(4-Allylaminomethyl-2-aminomethyl-benzyl)-(1H-benzoimidazol-2-ylmethyl)-(5,6,7,8-tetrahydro-quinolin-8-yl)-amine;
(2-Allylaminomethyl-4-aminomethyl-benzyl)-(1H-benzoimidazol-2-ylmethyl)-(5,6,7,8-tetrahydro-quinolin-8-yl)-amine;
(2-Aminomethyl-4-cyclopropylaminomethyl-benzyl)-(1H-benzoimidazol-2-ylmethyl)-(5,6,7,8-tetrahydro-quinolin-8-yl)-amine;
(4-Aminomethyl-2-cyclopropylaminomethyl-benzyl)-(1H-benzoimidazol-2-ylmethyl)-(5,6,7,8-tetrahydro-quinolin-8-yl)-amine;
(2-Aminomethyl-5-chloro-benzyl)-(1H-benzoimidazol-2-ylmethyl)-(5,6,7,8-tetrahydro-quinolin-8-yl)-amine;
(2-Aminomethyl-5-bromo-benzyl)-(1H-benzoimidazol-2-ylmethyl)-(5,6,7,8-tetrahydro-quinolin-8-yl)-amine;
(2-Aminomethyl-5-nitro-benzyl)-(1H-benzoimidazol-2-ylmethyl)-(5,6,7,8-tetrahydro-quinolin-8-yl)-amine;
4-Aminomethyl-3-{[(1H-benzoimidazol-2-ylmethyl)-(5,6,7,8-tetrahydro-quinolin-8-yl)-amino]-methyl}-benzonitrile;
(5-Amino-2-aminomethyl-benzyl)-(1H-benzoimidazol-2-ylmethyl)-(5,6,7,8-tetrahydro-quinolin-8-yl)-amine;
(2-Aminomethyl-5-trifluoromethyl-benzyl)-(1H-benzoimidazol-2-ylmethyl)-(5,6,7,8-tetrahydro-quinolin-8-yl)-amine;
(2-Aminomethyl-4-fluoro-benzyl)-(1H-benzoimidazol-2-ylmethyl)-(5,6,7,8-tetrahydro-quinolin-8-yl)-amine;
(2-Aminomethyl-4-chloro-benzyl)-(1H-benzoimidazol-2-ylmethyl)-(5,6,7,8-tetrahydro-quinolin-8-yl)-amine;
(2-Aminomethyl-4-bromo-benzyl)-(1H-benzoimidazol-2-ylmethyl)-(5,6,7,8-tetrahydro-quinolin-8-yl)-amine;
(2-Aminomethyl-4-nitro-benzyl)-(1H-benzoimidazol-2-ylmethyl)-(5,6,7,8-tetrahydro-quinolin-8-yl)-amine;
3-Aminomethyl-4-{[(1H-benzoimidazol-2-ylmethyl)-(5,6,7,8-tetrahydro-quinolin-8-yl)-amino]-methyl}-benzonitrile;
(4-Amino-2-aminomethyl-benzyl)-(1H-benzoimidazol-2-ylmethyl)-(5,6,7,8-tetrahydro-quinolin-8-yl)-amine;
(2-Aminomethyl-4-trifluoromethyl-benzyl)-(1H-benzoimidazol-2-ylmethyl)-(5,6,7,8-tetrahydro-quinolin-8-yl)-amine;
(4-Aminomethyl-2-fluoro-benzyl)-(1H-benzoimidazol-2-ylmethyl)-(5,6,7,8-tetrahydro-quinolin-8-yl)-amine;
(4-Aminomethyl-2-chloro-benzyl)-(1H-benzoimidazol-2-ylmethyl)-(5,6,7,8-tetrahydro-quinolin-8-yl)-amine;
(4-Aminomethyl-2-bromo-benzyl)-(1H-benzoimidazol-2-ylmethyl)-(5,6,7,8-tetrahydro-quinolin-8-yl)-amine;
(4-Aminomethyl-2-nitro-benzyl)-(1H-benzoimidazol-2-ylmethyl)-(5,6,7,8-tetrahydro-quinolin-8-yl)-amine;
5-Aminomethyl-2-{[(1H-benzoimidazol-2-ylmethyl)-(5,6,7,8-tetrahydro-quinolin-8-yl)-amino]-methyl}-benzonitrile;
(2-Amino-4-aminomethyl-benzyl)-(1H-benzoimidazol-2-ylmethyl)-(5,6,7,8-tetrahydro-quinolin-8-yl)-amine;
(4-Aminomethyl-2-trifluoromethyl-benzyl)-(1H-benzoimidazol-2-ylmethyl)-(5,6,7,8-tetrahydro-quinolin-8-yl)-amine;
(5-Aminomethyl-thiophen-2-ylmethyl)-(1H-benzoimidazol-2-ylmethyl)-(5,6,7,8-tetrahydro-quinolin-8-yl)-amine;
(4-Aminomethyl-thiophen-3-ylmethyl)-(1H-benzoimidazol-2-ylmethyl)-(5,6,7,8-tetrahydro-quinolin-8-yl)-amine;
(4-Aminomethyl-furan-3-ylmethyl)-(1H-benzoimidazol-2-ylmethyl)-(5,6,7,8-tetrahydro-quinolin-8-yl)-amine;
(4-Aminomethyl-1H-pyrrol-3-ylmethyl)-(1H-benzoimidazol-2-ylmethyl)-(5,6,7,8-tetrahydro-quinolin-8-yl)-amine;
(4-Aminomethyl-1-methyl-1H-pyrrol-3-ylmethyl)-(1H-benzoimidazol-2-ylmethyl)-(5,6,7,8-tetrahydro-quinolin-8-yl)-amine;
(4-Aminomethyl-1H-pyrazol-3-ylmethyl)-(1H-benzoimidazol-2-ylmethyl)-(5,6,7,8-tetrahydro-quinolin-8-yl)-amine;
(4-Aminomethyl-1-methyl-1H-pyrazol-3-ylmethyl)-(1H-benzoimidazol-2-ylmethyl)-(5,6,7,8-tetrahydro-quinolin-8-yl)-amine;
(3-Aminomethyl-1H-pyrazol-4-ylmethyl)-(1H-benzoimidazol-2-ylmethyl)-(5,6,7,8-tetrahydro-quinolin-8-yl)-amine;
(3-Aminomethyl-1-methyl-1H-pyrazol-4-ylmethyl)-(1H-benzoimidazol-2-ylmethyl)-(5,6,7,8-tetrahydro-quinolin-8-yl)-amine;
(5-Aminomethyl-3H-imidazol-4-ylmethyl)-(1H-benzoimidazol-2-ylmethyl)-(5,6,7,8-tetrahydro-quinolin-8-yl)-amine;
(5-Aminomethyl-1-methyl-1H-imidazol-4-ylmethyl)-(1H-benzoimidazol-2-ylmethyl)-(5,6,7,8-tetrahydro-quinolin-8-yl)-amine;
(5-Aminomethyl-thiazol-4-ylmethyl)-(1H-benzoimidazol-2-ylmethyl)-(5,6,7,8-tetrahydro-quinolin-8-yl)-amine;
(5-Aminomethyl-pyrimidin-4-ylmethyl)-(1H-benzoimidazol-2-ylmethyl)-(5,6,7,8-tetrahydro-quinolin-8-yl)-amine;
(5-Aminomethyl-pyridazin-4-ylmethyl)-(1H-benzoimidazol-2-ylmethyl)-(5,6,7,8-tetrahydro-quinolin-8-yl)-amine;
(5-Allylaminomethyl-2-{[(1H-benzoimidazol-2-ylmethyl)-(5,6,7,8-tetrahydro-quinolin-8-yl)-amino]-methyl}-phenyl)-methanol;
(3-Allylaminomethyl-4-{[(1H-benzoimidazol-2-ylmethyl)-(5,6,7,8-tetrahydro-quinolin-8-yl)-amino]-methyl}-phenyl)-methanol;
(4-Allylaminomethyl-2-methoxymethyl-benzyl)-(1H-benzoimidazol-2-ylmethyl)-(5,6,7,8-tetrahydro-quinolin-8-yl)-amine;
(3-Allylaminomethyl-4-methoxymethyl-benzyl)-(1H-benzoimidazol-2-ylmethyl)-(5,6,7,8-tetrahydro-quinolin-8-yl)-amine;
(2-{[(1H-Benzoimidazol-2-ylmethyl)-(5,6,7,8-tetrahydro-quinolin-8-yl)-amino]-methyl}-5-cyclopropylaminomethyl-phenyl)-methanol;
(4-{[(1H-Benzoimidazol-2-ylmethyl)-(5,6,7,8-tetrahydro-quinolin-8-yl)-amino]-methyl}-3-cyclopropylaminomethyl-phenyl)-methanol;
(1H-Benzoimidazol-2-ylmethyl)-(4-cyclopropylaminomethyl-2-methoxymethyl-benzyl)-(5,6,7,8-tetrahydro-quinolin-8-yl)-amine;
(1H-Benzoimidazol-2-ylmethyl)-(2-cyclopropylaminomethyl-4-methoxymethyl-benzyl)-(5,6,7,8-tetrahydro-quinolin-8-yl)-amine;
5-Aminomethyl-2-{[(1H-benzoimidazol-2-ylmethyl)-(5,6,7,8-tetrahydro-quinolin-8-yl)-amino]-methyl}-benzamide;
5-Aminomethyl-2-{[(1H-benzoimidazol-2-ylmethyl)-(5,6,7,8-tetrahydro-quinolin-8-yl)-amino]-methyl}-N-hydroxy-benzamide;
5-Aminomethyl-2-{[(1H-benzoimidazol-2-ylmethyl)-(5,6,7,8-tetrahydro-quinolin-8-yl)-amino]-methyl}-benzoic acid hydrazide;
5-Aminomethyl-2-{[(1H-benzoimidazol-2-ylmethyl)-(5,6,7,8-tetrahydro-quinolin-8-yl)-amino]-methyl}-benzoic acid;
(1H-Benzoimidazol-2-ylmethyl)-(2,4-bis-allylaminomethyl-benzyl)-(5,6,7,8-tetrahydro-quinolin-8-yl)-amine;
(4-Allylaminomethyl-2-cyclopropylaminomethyl-benzyl)-(1H-benzoimidazol-2-ylmethyl)-(5,6,7,8-tetrahydro-quinolin-8-yl)-amine;
(2-Allylaminomethyl-4-cyclopropylaminomethyl-benzyl)-(1H-benzoimidazol-2-ylmethyl)-(5,6,7,8-tetrahydro-quinolin-8-yl)-amine;
(1H-Benzoimidazol-2-ylmethyl)-(2,4-bis-cyclopropylaminomethyl-benzyl)-(5,6,7,8-tetrahydro-quinolin-8-yl)-amine;
(2-Aminomethyl-4-propyl-benzyl)-(1H-benzoimidazol-2-ylmethyl)-(5,6,7,8-tetrahydro-quinolin-8-yl)-amine;
(4-Allyl-2-aminomethyl-benzyl)-(1H-benzoimidazol-2-ylmethyl)-(5,6,7,8-tetrahydro-quinolin-8-yl)-amine;
Acetic acid 3-aminomethyl-4-{[(1H-benzoimidazol-2-ylmethyl)-(5,6,7,8-tetrahydro-quinolin-8-yl)-amino]-methyl}-benzyl ester;
Acetic acid 5-aminomethyl-2-{[(1H-benzoimidazol-2-ylmethyl)-(5,6,7,8-tetrahydro-quinolin-8-yl)-amino]-methyl}-benzyl ester;
Acetic acid 4-{[(1H-benzoimidazol-2-ylmethyl)-(5,6,7,8-tetrahydro-quinolin-8-yl)-amino]-methyl}-3-cyclopropylaminomethyl-benzyl ester;
Acetic acid 2-{[(1H-benzoimidazol-2-ylmethyl)-(5,6,7,8-tetrahydro-quinolin-8-yl)-amino]-methyl}-5-cyclopropylaminomethyl-benzyl ester;
Acetic acid 3-allylaminomethyl-4-{[(1H-benzoimidazol-2-ylmethyl)-(5,6,7,8-tetrahydro-quinolin-8-yl)-amino]-methyl}-benzyl ester;
Acetic acid 5-allylaminomethyl-2-{[(1H-benzoimidazol-2-ylmethyl)-(5,6,7,8-tetrahydro-quinolin-8-yl)-amino]-methyl}-benzyl ester;
5-Aminomethyl-2-{[(1H-benzoimidazol-2-ylmethyl)-(5,6,7,8-tetrahydro-quinolin-8-yl)-amino]-methyl}-benzaldehyde oxime;
3-Aminomethyl-4-{[(1H-benzoimidazol-2-ylmethyl)-(5,6,7,8-tetrahydro-quinolin-8-yl)-amino]-methyl}-benzaldehyde oxime;
N-(5-Aminomethyl-2-{[(1H-benzoimidazol-2-ylmethyl)-(5,6,7,8-tetrahydro-quinolin-8-yl)-amino]-methyl}-benzyl)-acetamide;
N-(3-Aminomethyl-4-{[(1H-benzoimidazol-2-ylmethyl)-(5,6,7,8-tetrahydro-quinolin-8-yl)-amino]-methyl}-benzyl)-acetamide;
N-(3-(Acetylamino-methyl)-4-{[(1H-benzoimidazol-2-ylmethyl)-(5,6,7,8-tetrahydro-quinolin-8-yl)-amino]-methyl}-benzyl)-acetamide;
N-(2-{[(1H-Benzoimidazol-2-ylmethyl)-(5,6,7,8-tetrahydro-quinolin-8-yl)-amino]-methyl}-benzyl)-acetamide;
(6-Aminomethyl-1,3-dihydro-isobenzofuran-5-ylmethyl)-(1H-benzoimidazol-2-ylmethyl)-(5,6,7,8-tetrahydro-quinolin-8-yl)-amine;
(4-Aminomethyl-1,3-dihydro-isobenzofuran-5-ylmethyl)-(1H-benzoimidazol-2-ylmethyl)-(5,6,7,8-tetrahydro-quinolin-8-yl)-amine;
(7-Aminomethyl-1,3-dihydro-isobenzofuran-4-ylmethyl)-(1H-benzoimidazol-2-ylmethyl)-(5,6,7,8-tetrahydro-quinolin-8-yl)-amine;
N′-(1H-benzimidazol-2-ylmethyl)-N′-(5,6,7,8-tetrahydro-8-quinolinyl)-1,3-benzenedimethanamine;
(1H-Benzimidazol-2-ylmethyl)-(2-Aminomethyl-benzyl)-(5,6,7,8-tetrahydro-quinolin-8-yl)-amine;
(2-Aminomethyl-benzyl)-(1H-benzimidazol-2-ylmethyl)-(S)-5,6,7,8-tetrahydro-quinolin-8-yl-amine;
(3-aminomethyl-4-{[(1H-benzoimidazol-2-ylmethyl)-(5,6,7,8-tetrahydro-quinolin-8-yl)-amino]-methyl}-phenyl)-methanol;
(2-Aminomethyl-3-methoxy-benzyl)-(1H-benzimidazol-2-ylmethyl)-(5,6,7,8-tetrahydro-quinolin-8-yl)-amine;
(1H-Benzimidazol-2-ylmethyl)-(5,6,7,8-tetrahydroquinolin-8-yl)-[(1-aminomethyl)-benzoxazol-3-ylmethyl)]-amine;
(1H-Benzimidazol-2-ylmethyl)-(5,6,7,8-tetrahydroquinolin-8-yl)-[(1-benzyl-2-aminomethyl)-imidazol-5-ylmethyl)]-amine;
6-aminomethylpyridin-3-ylmethyl-(1H-benzimidazol-2-ylmethyl)-(5,6,7,8-tetrahydroquinolin-8-yl)-amine;
[4-(2-amino-ethyl)-benzyl]-(1H-benzoimidazol-2-ylmethyl)-(5,6,7,8-tetrahydro-quinolin-8-yl)-amine;
[4-(3-amino-propyl)-benzyl]-(1H-benzoimidazol-2-ylmethyl)-(5,6,7,8-tetrahydro-quinolin-8-yl)-amine;
N-(4-{[(1H-benzimidazol-2-ylmethyl)-(5,6,7,8-tetrahydro-quinolin-8-yl)-amino]-methyl}-benzyl)-hydroxylamine;
(5-aminomethyl-2-{[(1H-benzoimidazol-2-ylmethyl)-(5,6,7,8-tetrahydro-quinolin-8-yl)-amino]-methyl}-phenyl)-methanol;
2-Aminomethyl-5-{[(1H-benzimidazol-2-ylmethyl)-(5,6,7,8-tetrahydro-quinolin-8-yl)-amino]-methyl}-phenol;
(4-Aminomethyl-3-methoxy-benzyl)-(1H-benzimidazol-2-ylmethyl)-(5,6,7,8-tetrahydro-quinolin-8-yl)-amine;
(1H-benzoimidazol-2-ylmethyl)-(2,4-bis-aminomethyl-benzyl)-(5,6,7,8-tetrahydro-quinolin-8-yl)-amine;
5-Aminomethyl-2-{[(1H-benzoimidazol-2-ylmethyl)-(5,6,7,8-tetrahydro-quinolin-8-yl)-amino]-methyl}-benzoic acid methyl ester;
3-aminomethyl-4-{[(1H-benzimidazole-2-ylmethyl)-(5,6,7,8-tetrahydro-quinolin-8-yl)-amino]-methyl}-benzoic acid;
3-aminomethyl-4-{[(1H-benzimidazole-2-ylmethyl)-(5,6,7,8-tetrahydro-quinolin-8-yl)-amino]-methyl}-N-hydroxy-benzamide;
3-aminomethyl-4-{[(1H-benzimidazole-2-ylmethyl)-(5,6,7,8-tetrahydro-quinolin-8-yl)-amino]-methyl}-benzamide;
3-Aminomethyl-4-{[(1H-benzimidazole-2-ylmethyl)-(5,6,7,8-tetrahydro-quinolin-8-yl)-amino]-methyl}-benzoic acid hydrazide;
(2-aminomethyl-5-fluorobenzyl)-(1H-benzimidazol-2-ylmethyl)-(5,6,7,8-tetrahydro-quinolin-8-yl)-amine;
3-aminomethyl-4-{[(1H-benzimidazol-2-ylmethyl)-(5,6,7,8-tetrahydro-quinolin-8-yl)-amino]-methyl}-benzoic acid methyl ester;
(2-aminomethyl-4-methoxymethyl-benzyl)-(1H-benzimidazol-2-ylmethyl)-(5,6,7,8-tetrahydro-quinolin-8-yl)-amine;
N-(2-{[(1H-benzimidazol-2-ylmethyl)-(5,6,7,8-tetrahydroquinolin-8-yl)-amino]-methyl}-benzyl)-guanidine;
N-(4-{[(1H-benzimidazol-2-ylmethyl)-(5,6,7,8-tetrahydroquinolin-8-yl)-amino]-methyl}-benzyl)-guanidine;
N′-(4-{[(1H-Benzimidazol-2-ylmethyl)-(5,6,7,8-tetrahydro-quinolin-8-yl)-amino]-methyl}-benzyl)-N,N-dimethyl-guanidine;
[4-(1H-benzimidazol-2-ylmethyl)-(5,6,7,8-tetrahydroquinolin-8-yl)-aminomethylbenzyl]-N,N-dimethylformamidine;
N-(4-{[(1H-Benzimidazol-2-ylmethyl)-(5,6,7,8-tetrahydro-quinolin-8-yl)-amino]-methyl}-benzyl)-benzamidine;
N-(4-{[(1H-Benzimidazol-2-ylmethyl)-(5,6,7,8-tetrahydro-quinolin-8-yl)-amino]-methyl}-benzyl)-acetamidine;
N-isobutyl-N′-(1H-benzimidazol-2-ylmethyl)-N′-(5,6,7,8-tetrahydro-8-quinolinyl)-1,4-benzenedimethanamine;
(1H-Benzimidazol-2-ylmethyl)-(4-piperidin-2-yl-benzyl)-(5,6,7,8-tetrahydro-quinolin-8-yl)-amine;
(1H-Benzimidazol-2-ylmethyl)-(4-piperidin-1-ylmethyl-benzyl)-(5,6,7,8-tetrahydro-quinolin-8-yl)-amine;
(1H-Benzimidazol-2-ylmethyl)-(4-methylaminomethyl-benzyl)-(5,6,7,8-tetrahydro-quinolin-8-yl)-amine;
(1H-Benzimidazol-2-ylmethyl)-(4-piperazin-1-ylmethyl-benzyl)-(5,6,7,8-tetrahydro-quinolin-8-yl)-amine;
[4-(4-Allyl-piperazin-1-ylmethyl)-benzyl]-(1H-benzimidazol-2-ylmethyl)-(5,6,7,8-tetrahydro-quinolin-8-yl)-amine;
(1H-Benzimidazol-2-ylmethyl)-(4-dimethylaminomethyl-benzyl)-(5,6,7,8-tetrahydro-quinolin-8-yl)-amine;
(1H-Benzimidazol-2-ylmethyl)-(5,6,7,8-tetrahydro-quinolin-8-yl)-[4-(1,2,4-triazol-4-yliminomethyl)-benzyl]-amine;
N′-(4-{[(1H-Benzimidazol-2-ylmethyl)-(5,6,7,8-tetrahydro-quinolin-8-yl)-amino]-methyl}-benzyl)-ethane-1,2-diamine;
(1H-benzimidazol-2-ylmethyl)-(4-butylaminomethyl-benzyl)-(5,6,7,8-tetrahydro-quinolin-8-yl)-amine;
(1H-benzimidazol-2-ylmethyl)-(4-diallylaminomethyl-benzyl)-(5,6,7,8-tetrahydro-quinolin-8-yl)-amine;
(4-allylaminomethyl-benzyl)-(1H-benzimidazol-2-ylmethyl)-(5,6,7,8-tetrahydro-quinolin-8-yl)-amine;
(1H-benzimidazol-2-ylmethyl)-(4-pyrrolidin-1-ylmethyl-benzyl)-(5,6,7,8-tetrahydro-quinolin-8-yl)-amine;
(1H-Benzimidazol-2-ylmethyl)-(4-morpholin-4-ylmethyl-benzyl)-(5,6,7,8-tetrahydro-quinolin-8-yl)-amine;
(1H-benzoimidazol-2-ylmethyl)-(5,6,7,8-tetrahydro-quinolin-8-yl)-(4-thiomorpholin-4-ylmethyl-benzyl)-amine;
(1H-Benzimidazol-2-ylmethyl)-(5,6,7,8-tetrahydro-quinolin-8-yl)-(2-cyclopropylaminomethyl-benzyl)-amine;
(1H-Benzimidazol-2-ylmethyl)-(5,6,7,8-tetrahydro-quinolin-8-yl)-(2-allylaminomethyl-benzyl)-amine;
(1H-Benzimidazol-2-ylmethyl)-[2-(R)-(2-aminopropionamidylmethyl)-benzyl]-(5,6,7,8-tetrahydro-quinolin-8-yl)-amine;
(1H-benzimidazol-2-ylmethyl)-[2-(1H-benzimidazol-2-ylmethyl)-aminobenzyl]-(5,6,7,8-tetrahydroquinolin-8-yl)-amine;
(2-aminobenzyl)-(1H-benzimidazol-2-ylmethyl)-(5,6,7,8-tetrahydroquinolin-8-yl)-amine;
(1H-Benzimidazol-2-ylmethyl)-(2-cyano-benzyl)-(5,6,7,8-tetrahydro-quinolin-8-yl)-amine;
2-{[(1H-Benzimidazol-2-ylmethyl)-(5,6,7,8-tetrahydro-quinolin-8-yl)-amino]-methyl}-6-methoxy-benzoic acid ethyl ester;
(6-aminopyridin-3-ylmethyl)-(benzimidazol-2-ylmethyl)-(5,6,7,8-tetrahydroquinolin-8-yl)-amine;
(2-aminopyridin-3-ylmethyl)-(benzimidazol-2-ylmethyl)-(5,6,7,8-tetrahydro-8-quinolinyl)-amine;
N-(4-{[(1H-Benzimidazol-2-ylmethyl)-(5,6,7,8-tetrahydro-quinolin-8-yl)-amino]-methyl}-phenyl)-guanidine;
(4-Amino-benzyl)-(1H-benzoimidazol-2-ylmethyl)-(5,6,7,8-tetrahydro-quinolin-8-yl)-amine;
N′-({[(1H-Benzimidazol-2-ylmethyl)-(5,6,7,8-tetrahydroquinolin-8-yl)-amino]-methyl}-phenyl)-N,N-dimethylformamidine;
4-{[(1H-Benzimidazol-2-ylmethyl)-(5,6,7,8-tetrahydro-quinolin-8-yl)-amino]-methyl}-benzaldehyde oxime;
[4-(1H-benzimidazol-2-ylmethyl)-(5,6,7,8-tetrahydroquinolin-8-yl)-aminomethyl]-benzamidine;
4-{[(1H-Benzimidazol-2-ylmethyl)-(5,6,7,8-tetrahydro-quinolin-8-yl)-amino]-methyl}-benzyl alcohol;
4-{[(1H-Benzimidazol-2-ylmethyl)-(5,6,7,8-tetrahydro-quinolin-8-yl)-amino]-methyl}-benzaldehyde;
4-{[(1H-Benzimidazol-2-ylmethyl)-(5,6,7,8-tetrahydro-quinolin-8-yl)-amino]-methyl}-benzoic acid methyl ester;
(R,S)-4-{[(1H-Benzimidazol-2-ylmethyl)-(5,6,7,8-tetrahydro-quinolin-8-yl)-amino]-methyl}-N-hydroxy-benzamide;
4-{[(1H-Benzimidazol-2-ylmethyl)-(5,6,7,8-tetrahydro-quinolin-8-yl)-amino]-methyl}-benzoic acid hydrazide;
4-{[(1H-benzoimidazol-2-ylmethyl)-(5,6,7,8-tetrahydro-quinolin-8-yl)-amino]-methyl}-benzoic acid;
4-{[(1H-Benzimidazol-2-ylmethyl)-(5,6,7,8-tetrahydro-quinolin-8-yl)-amino]-methyl}-benzamide;
(6-Amino-pyridin-2-ylmethyl)-(1H-benzimidazol-2-ylmethyl)-(5,6,7,8-tetrahydro-quinolin-8-yl)-amine;
(2-{[(1H-Benzimidazol-2-ylmethyl)-(5,6,7,8-tetrahydro-quinolin-8-yl)-amino]-methyl}-phenyl)-methanol;
O-(2-{[(1H-Benzimidazol-2-ylmethyl)-(5,6,7,8-tetrahydro-quinolin-8-yl)-amino]-methyl}-benzyl)-hydroxylamine;
(4-Amino-pyridin-3-ylmethyl)-(1H-benzimidazol-2-ylmethyl)-(5,6,7,8-tetrahydro-quinolin-8-yl)-amine;
2-{[(1H-Benzimidazol-2-ylmethyl)-(5,6,7,8-tetrahydro-quinolin-8-yl)-amino]-methyl}-5-cyano-benzoic acid methyl ester;
4-{[(1H-benzimidazol-2-ylmethyl)-(5,6,7,8-tetrahydroquinolin-8-yl)-amino]-methyl}-3-cyano-benzamide;
[3-(1H-benzimidazol-2-yl)-benzyl]-(1H-benzimidazol-2-ylmethyl)-(5,6,7,8-tetrahydroquinolin-8-yl)-amine;
(1H-Benzimidazol-2-ylmethyl)-(5,6,7,8-tetrahydroquinolin-8-yl)-(imidazol-2-yl)-methyl amine;
4-{[(1H-Benzimidazol-2-ylmethyl)-(5,6,7,8-tetrahydro-quinolin-8-yl)-amino]-methyl}-2,6-dichloropyridine;
(1H-benzoimidazol-2-ylmethyl)-benzooxazol-5-ylmethyl-(5,6,7,8-tetrahydro-quinolin-8-yl)-amine;
pyridin-2-ylmethyl-(1H-benzimidazol-2-ylmethyl)-(5,6,7,8-tetrahydroquinolin-8-yl)-amine;
(1H-benzimidazol-2-ylmethyl)-benzoxazol-6-ylmethyl-(5,6,7,8-tetrahydro-quinolin-8-yl)-amine;
(1H-benzimidazol-4-ylmethyl)-(1H-benzimidazol-2-ylmethyl)-(5,6,7,8-tetrahydro-quinolin-8-yl)-amine;
(1H-Benzimidazol-2-ylmethyl)-pyridin-4-ylmethyl-(5,6,7,8-tetrahydro-quinolin-8-yl)-amine;
(1H-Benzimidazol-2-ylmethyl)-(benzo[1,3]-dioxol-4-ylmethyl)-(5,6,7,8-tetrahydro-quinolin-8-yl)-amine;
benzo[1,3]dioxol-5-ylmethyl-(1H-benzoimidazol-2-ylmethyl)-(5,6,7,8-tetrahydro-quinolin-8-yl)-amine;
(1H-Benzimidazol-2-ylmethyl)-(2,3-dihydro-benzofuran-7-ylmethyl)-(5,6,7,8-tetrahydro-quinolin-8-yl)-amine;
(1H-Benzimidazol-2-ylmethyl)-pyridin-3-ylmethyl-(5,6,7,8-tetrahydro-quinolin-8-yl)-amine;
(1H-benzoimidazol-5-ylmethyl)-(1H-benzoimidazol-2-ylmethyl)-(5,6,7,8-tetrahydro-quinolin-8-yl)-amine;
Bis-(1H-benzimidazol-2-ylmethyl)-(5,6,7,8-tetrahydro-quinolin-8-yl)-amine;
(1H-Benzimidazol-2-ylmethyl)-(3H-imidazol-4-ylmethyl)-(5,6,7,8-tetrahydro-quinolin-8-yl)-amine;
[4-(1H-benzimidazol-2-yl)-benzyl]-(1H-benzimidazol-2-ylmethyl)-(5,6,7,8-tetrahydroquinolin-8-yl)-amine;
(1H-Benzimidazol-2-ylmethyl)-(4-pyrid-2-yl-benzyl)-(5,6,7,8-tetrahydro-quinolin-8-yl)-amine;
(1H-Benzimidazol-2-ylmethyl)-[4-(oxazol-2-yl)-benzyl]-(5,6,7,8-tetrahydroquinolin-8-yl)-amine;
(1H-Benzimidazol-2-ylmethyl)-(4-imidazol-1-yl-benzyl)-(5,6,7,8-tetrahydro-quinolin-8-yl)-amine;
[4-(thiazol-2-yl)-benzyl]-(1H-benzimidazol-2-ylmethyl)-(5,6,7,8-tetrahydroquinolin-8-yl)-amine;
(1H-Benzimidazol-2-ylmethyl)-[4-(benzothiazol-2-yl)-benzyl]-(5,6,7,8-tetrahydroquinolin-8-yl)-amine;
[4-(benzoxazol-2-yl)-benzyl]-(1H-benzimidazol-2-ylmethyl)-(5,6,7,8-tetrahydroquinolin-8-yl)-amine;
[4-(1H-imidazol-2-yl)-benzyl]-(1H-benzimidazol-2-ylmethyl)-(5,6,7,8-tetrahydroquinolin-8-yl)-amine;
(2′-Aminomethyl-biphenyl-4-ylmethyl)-(1H-benzimidazol-2-ylmethyl)-(5,6,7,8-tetrahydro-quinolin-8-yl)-amine;
(1H-Benzimidazol-2-ylmethyl)-(2′-methoxy-biphenyl-4-ylmethyl)-(5,6,7,8-tetrahydro-quinolin-8-yl)-amine;
(1H-Benzimidazol-2-ylmethyl)-(4-oxazol-5-yl-benzyl)-(5,6,7,8-tetrahydro-quinolin-8-yl)-amine;
(1H-Benzimidazol-2-ylmethyl)-(5,6,7,8-tetrahydro-quinolin-8-yl)-(4-thiophen-2-yl-benzyl)-amine;
(1H-Benzimidazol-2-ylmethyl)-[4-(2-methyl-2H-tetrazol-5-yl)-benzyl]-(5,6,7,8-tetrahydro-quinolin-8-yl)-amine;
(1H-Benzimidazol-2-ylmethyl)-[4-(5-phenyloxazol-2-yl)-benzyl]-(5,6,7,8-tetrahydroquinolin-8-yl)-amine;
N¹-(1H-Benzimidazol-2-ylmethyl)-N¹-(5,6,7,8-tetrahydro-quinolin-8-yl)-ethane-1,2-diamine;
N¹-(1H-Benzimidazol-2-ylmethyl)-N²-(5-nitro-pyridin-2-yl)-N¹-(5,6,7,8-tetrahydro-quinolin-8-yl)-ethane-1,2-diamine;
N-(6-2-[(1H-Benzimidazol-2-ymethyl)-(5,6,7,8-tetrahydro-quinolin-8-yl)-amino]-ethylamino}-pyridin-3-yl)-acetamide;
N¹-(1H-Benzimidazol-2-ylmethyl)-N¹-(5,6,7,8-tetrahydro-quinolin-8-yl)-propane-1,3-diamine;
N¹-(1H-Benzimidazol-2-ylmethyl)-N²-pyridin-2-ylmethyl-N¹-(5,6,7,8-tetrahydro-quinolin-8-yl)-ethane-1,2-diamine;
N¹-(1H-Benzimidazol-2-ylmethyl)-N¹-(5,6,7,8-tetrahydro-quinolin-8-yl)-butane-1,4-diamine;
N′-(1H-benzimidazol-2-ylmethyl)-N′-(S)-5,6,7,8-tetrahydro-quinolin-8-yl-butane-1,4-diamine;
N¹-(1-Methyl-1H-benzoimidazol-2-ylmethyl)-N¹-(5,6,7,8-tetrahydro-quinolin-8-yl)-butane-1,4-diamine;
N¹-[5-(4-Fluoro-phenyl)-1H-imidazol-2-ylmethyl]-N¹-(5,6,7,8-tetrahydro-quinolin-8-yl)-butane-1,4-diamine;
N′-(1H-Benzimidazol-2-ylmethyl)-(5,6,7,8-tetrahydroquinolin-8-yl)-N-benzyl-1,4-butanediamine;
N¹-(1H-Benzimidazol-2-ylmethyl)-N⁴-pyridin-2-ylmethyl-N¹-(5,6,7,8-tetrahydro-quinolin-8-yl)-butane-1,4-diamine;
N¹-(1H-Benzimidazol-2-ylmethyl)-N⁴-(1H-indol-3-ylmethyl)-N¹-(5,6,7,8-tetrahydro-quinolin-8-yl)-butane-1,4-diamine;
1-N′-[4-(1H-Benzimidazol-2-ylmethyl)-(5,6,7,8-tetrahydroquinolin-8-yl)-amino]-aminobutane-N,N-dimethylformamidine;
N-{4-[(1H-benzoimidazol-2-ylmethyl)-(5,6,7,8-tetrahydro-quinolin-8-yl)-amino]-butyl}-guanidine;
N′-(1H-benzimidazol-2-ylmethyl)-N′-(5,6,7,8-tetrahydro-quinolin-8-yl)-butane-1,4-diamine-N-(2-pyridinyl)-sulfonamide;
N-(1H-benzoimidazol-2-ylmethyl)-N′-pyrimidin-2-ylmethyl-N-(5,6,7,8-tetrahydro-quinolin-8-yl)-butane-1,4-diamine;
N-(1H-benzoimidazol-2-ylmethyl)-N′-(1H-imidazol-2-yl)-N-(5,6,7,8-tetrahydro-quinolin-8-yl)-butane-1,4-diamine;
N¹-(1H-benzimidazol-2-ylmethyl)-N⁴-(1H-indol-2-ylmethyl)-N¹-(5,6,7,8-tetrahydroquinolin-8-yl)-butane-1,4-diamine;
(1H-Benzimidazol-2-ylmethyl)-(5,6,7,8-tetrahydroquinolin-8-yl)-(N,N-dimethyl-4-amino-but-1-yl)-amine;
(1H-Benzimidazol-2-ylmethyl)-(5,6,7,8-tetrahydroquinolin-8-yl)-(N-allyl-4-amino-but-1-yl)-amine;
(1H-Benzimidazol-2-ylmethyl)-(5,6,7,8-tetrahydroquinolin-8-yl)-(N-methyl-4-amino-but-1-yl)-amine;
N′-(1H-imidazol-2-ylmethyl)-N′-(5,6,7,8-tetrahydro-quinolin-8-yl)-butane-1,4-diamine;
N-{4-[(1H-benzoimidazol-2-ylmethyl)-(5,6,7,8-tetrahydro-quinolin-8-yl)-amino]-butyl}-benzenesulfonamide;
(2S)-2-Amino-5-[(1H-benzimidazol-2-ylmethyl)-(5,6,7,8-tetrahydroquinolin-8-yl)-amino]-pentanoic acid;
N¹-(1H-Benzimidazol-2-ylmethyl)-N⁴-cyclopropyl-N¹-(5,6,7,8-tetrahydro-quinolin-8-yl)-butane-1,4-diamine;
N′-(1H-benzimidazol-2-ylmethyl)-3-methyl-3-phenyl-N′-(5,6,7,8-tetrahydro-quinolin-8-yl)-butane-1,4-diamine;
(1H-benzimidazol-2-ylmethyl)-(5,6,7,8-tetrahydro-quinolin-8-yl)-(1-phenyl-1-aminobut-4-yl)-amine;
(1H-benzimidazol-2-ylmethyl)-(5,6,7,8-tetrahydro-quinolin-8-yl)-(1-aminobutan-3-ol-4-yl)-amine;
(1H-benzimidazol-2-ylmethyl)-(5,6,7,8-tetrahydro-quinolin-8-yl)-(1-amino-3-fluoro-butan-4-yl)-amine;
(1H-Benzimidazol-2-ylmethyl)-(5,6,7,8-tetrahydroquinolin-8-yl)-(5-amino-pent-1-yl)-amine;
(1H-Benzimidazol-2-ylmethyl)-(5,6,7,8-tetrahydroquinolin-8-yl)-(6-amino-hex-1-yl)-amine;
N¹-(1H-Benzimidazol-2-ylmethyl)-N¹—(R)-5,6,7,8-tetrahydro-quinolin-8-yl-butane-1,4-diamine;
N¹-(1H-Benzimidazol-2-ylmethyl)-2-methylene-N¹-(5,6,7,8-tetrahydro-quinolin-8-yl)-butane-1,4-diamine;
N¹-(1H-Benzimidazol-2-ylmethyl)-3-methoxy-N¹-(5,6,7,8-tetrahydro-quinolin-8-yl)-butane-1,4-diamine;
N¹-(1H-benzimidazol-2-ylmethyl)-3,3-difluoro-N¹-(5,6,7,8-tetrahydroquinolin-8-yl)-butane-1,4-diamine;
N¹-(1H-benzimidazol-2-ylmethyl)-2,2-difluoro-N¹-(5,6,7,8-tetrahydroquinolin-8-yl)-butane-1,4-diamine;
(1H-benzimidazol-2-ylmethyl)-(5,6,7,8-tetrahydro-quinolin-8-yl)-(1-amino-2-(O-methyloxime)-butan-4-yl)-amine;
(1H-benzimidazol-2-ylmethyl)-(5,6,7,8-tetrahydro-quinolin-8-yl)-(1-amino-2-methylenyl-butan-4-yl)-amine;
(1H-benzimidazol-4-methoxy-2-ylmethyl)-(5,6,7,8-tetrahydroquinolin-8-yl)-(1-aminobutan-4-yl)-amine;
N¹-(1H-Benzimidazol-2-ylmethyl)-N¹-(4-methoxy-5,6,7,8-tetrahydro-quinolin-8-yl)-butane-1,4-diamine;
N¹-(1H-benzimidazol-2-ylmethyl)-N¹⁻(3-methoxy-5,6,7,8-tetrahydroquinolin-8-yl)-butane-1,4-diamine;
N¹-(1-Methyl-1H-benzoimidazol-2-ylmethyl)-N¹—(S)-(5,6,7,8-tetrahydro-quinolin-8-yl)-butane-1,4-diamine;
N¹-(1H-benzimidazol-2-ylmethyl)-N¹-(2-chloro-5,6,7,8-tetrahydro-quinolin-8-yl)-butane-1,4-diamine;
N¹-(1H-Benzoimidazol-2-ylmethyl)-N¹-(2-methyl-5,6,7,8-tetrahydroquinolin-8-yl)-butane-1,4-diamine;
N¹-(5,6-dimethyl-1H-benzimidazol-2-ylmethyl)-N¹-(5,6,7,8-tetrahydro-quinolin-8-yl)-butane-1,4-diamine;
N¹-[1-(1H-Benzimidazol-2-yl)-ethyl]-N¹-(5,6,7,8-tetrahydro-quinolin-8-yl)-butane-1,4-diamine;
N¹-(4-fluoro-1H-benzimidazol-2-ylmethyl)-N¹-(5,6,7,8-tetrahydro-quinolin-8-yl)-butane-1,4-diamine;
N¹-(4-Methoxy-1H-benzoindiazol-2-ylmethyl)-N¹—(S)-(5,6,7,8-tetrahydro-quinolin-8-yl)-butane-1,4-diamine;
N¹-(4-methyl-1H-benzoimidazol-2-ylmethyl)-N¹-(5,6,7,8-tetrahydro-quinolin-8-yl)-butane-1,4-diamine;
N¹-(4,5-dimethyl-1H-benzoimidazol-2-ylmethyl)-N¹-(5,6,7,8-tetrahydro-quionlin-8-yl)-butane-1,4-diamine;
N¹-(6-Fluoro-1H-benzimidazol-2-ylmethyl)-N¹-(5,6,7,8-tetrahydroquinolin-8-yl)-butane-1,4-diamine;
N¹-(1H-imidazol-[4,5-b]pyridin-2-ylmethyl)-N¹-(5,6,7,8-tetrahydro-quinolin-8-yl)-butane-1,4-diamine;
N¹-(1H-imidazo[4,5-c]pyridin-2-ylmethyl)-N¹-(5,6,7,8-tetrahydro-quinolin-8-yl)-butane-1,4-diamine;
N¹-(5-trifluoromethyl-1H-benzoimidazol-2-ylmethyl)-N¹-(5,6,7,8-tetrahydro-quinolin-8-yl)-butane-1,4-diamine;
N¹-(1-allyl-1H-benzoimidazol-2-ylmethyl)-N¹-(5,6,7,8-tetrahydro-quinolin-8-yl)-butane-1,4-diamine;
N¹-(1-Allyl-1H-benzimidazol-2-ylmethyl)-N¹—(S)-5,6,7,8-tetrahydro-quinolin-8-yl-butane-1,4-diamine;
N¹-(1-cyclopropylmethyl-1H-benzoimidazol-2-ylmethyl)-N¹-(5,6,7,8-tetrahydro-quinolin-8-yl)-butane-1,4-diamine;
N¹-(1-pyridin-2-ylmethyl-1H-imidazol-2-ylmethyl)-N¹-(5,6,7,8-tetrahydro-quinolin-8-yl)-butane-1,4-diamine;
N¹-(4-methyl-1H-imidazol-2-ylmethyl)-N¹-(5,6,7,8-tetrahydro-quinolin-8-yl)-butane-1,4-diamine;
N¹-(1-isopropyl-1H-imidazol-2-ylmethyl)-N¹-(5,6,7,8-tetrahydro-quinolin-8-yl)-butane-1,4-diamine;
N¹-[1-(2-methoxy-ethyl)-1H-imidazol-2-ylmethyl]-N¹-(5,6,7,8-tetrahydro-quinolin-8-yl)-butane-1,4-diamine;
N¹-(4-methyl-1-propyl-1H-imidazol-2-ylmethyl)-N¹-(5,6,7,8-tetrahydro-quinolin-8-yl)-butane-1,4-diamine;
N¹-(1-propyl-1H-imidazol-2-ylmethyl)-N¹-(5,6,7,8-tetrahydroquinolin-8-yl)-butane-1,4-diamine;
N¹-(1-methyl-1H-imidazol-2-ylmethyl)-N¹-(5,6,7,8-tetrahydro-quinolin-8-yl)butane-1,4-diamine;
N¹-(1-allyl-1H-imidazol-2-ylmethyl)-N¹-(5,6,7,8-tetrahydroquinolin-8-yl)-butane-1,4-diamine;
N¹-(4-Methoxymethyl-1H-imidazol-2-ylmethyl)-N¹-(5,6,7,8-tetrahydro-quinolin-8-yl)-butane-1,4-diamine;
N¹-(1-Allyl-1H-imidazol-2-ylmethyl)-N¹—(S)-5,6,7,8-tetrahydro-quinolin-8-yl-butane-1,4-diamine;
N¹-{2-[(1H-Benzimidazol-2-ylmethyl)-N¹-(5,6,7,8-tetrahydro-quinolin-8-yl)-amino]-ethyl}-guanidine;
{4-[(1H-Benzimidazol-2-ylmethyl)-(5,6,7,8-tetrahydro-quinolin-8-yl)-amino]-butylamino}-acetic acid methyl ester;
pyrazine-2-carboxylic acid {4-[(1H-benzimidazol-2-ylmethyl)-(S)-5,6,7,8-tetrahydro-quinolin-8-yl-amino]-butyl}-amide;
Pyridine-2-carboxylic acid {3-[(1H-benzimidazol-2-ylmethyl)-(5,6,7,8-tetrahydro-quinolin-8-yl)-amino]-propyl}-amide;
Isoquinoline-3-carboxylic acid {3-[(1H-benzimidazol-2-ylmethyl)-(5,6,7,8-tetrahydro-quinolin-8-yl)-amino]-propyl}-amide;
N-{3-[(1H-benzoimidazol-2-ylmethyl)-(5,6,7,8-tetrahydro-quinolin-8-yl)-amino]-propyl}-6-hydroxy-nicotinamide;
N-3-[(1H-benzoimidazol-2-ylmethyl)-(5,6,7,8-tetrahydro-quinolin-8-yl)-amino]-propyl 1-benzamide;
N-{3-[(1H-benzoimidazol-2-ylmethyl)-(5,6,7,8-tetrahydro-quinolin-8-yl)-amino]-propyl}-5-bromo-nicotinamide;
Cinnoline-4-carboxylic acid-{3-[(1H-benzoimidazol-2-ylmethyl)-(5,6,7,8-tetrahydro-quinolin-8-yl)-amino]-propyl}-amide;
N-{4-[1H-benzolimidazol-2-ylmethyl)-(5,6,7,8-tetrahydroquinolin-8-yl)-amino]-butyl}-6-hydroxynicotinamide;
N-{4-[(1H-benzolimidazol-2-ylmethyl)-(5,6,7,8-tetrahydroquinolin-8-yl)-amino]-butyl}-benzamide;
pyridine-2-carboxylic acid {4-[1H-benzoimidazol-2-ylmethyl)-(5,6,7,8-tetrahydroquinolin-8-yl)-amino]-butyl}-amide;
N-{4-[(1H-Benzoimidazol-2-ylmethyl)-(5,6,7,8-tetrahydroquinolin-8-yl)-amino]-butyl}-5-bromo-nicotinamide;
quinoline-2-carboxylic acid {2-[(1H-benzoimidazol-2-ylmethyl)-(5,6,7,8-tetrahydroquinolin-8-yl)-amino]-butyl}-amide;
cinnoline-4-carboxylic acid {4-[(1H-benzoimidazol-2-ylmethyl)-5,6,7,8-tetrahydroquinolin-8-yl)-amino]-butyl}-amide;
N-{2-[(1H-benzoimidazol-2-ylmethyl)-(5,6,7,8-tetrahydro-quinolin-8-yl)-amino]-ethyl}-3,5-dichloro-isonicotinamide;
N-{3-[(1H-benzoimidazol-2-ylmethyl)-(5,6,7,8-tetrahydro-quinolin-8-yl)-amino]-propyl}-3,5-dichloro-isonicotinamide;
N-{4-[(1-allyl-1H-benzimidazol-2-ylmethyl)-(S)-5,6,7,8-tetrahydro-quinolin-8-ylamino]-butyl}-3,5-dichloro-isonicotinamide;
N-{4-[(1H-benzimidazol-2-ylmethyl)-(R)-5,6,7,8-tetrahydro-quinolin-8-yl-amino]-butyl}-3,5-dichloro-isonicotinamide;
N-{4-[(1-allyl-1H-imidazol-2-ylmethyl)-(S)-5,6,7,8-tetrahydro-quinolin-8-ylamino]-butyl}-3,5-dichloro-isonicotinamide;
N-{4-[(1H-Benzimidazol-2-ylmethyl)-(S)-5,6,7,8-tetrahydro-quinolin-8-yl-amino]-butyl}-acetamide;
{4-[(1H-Benzimidazol-2-ylmethyl)-(S)-5,6,7,8-tetrahydro-quinolin-8-yl-amino]-butyl}-urea;
pyrazine-2-carboxylic acid {3-[(1H-benzimidazol-2-ylmethyl)-(5,6,7,8-tetrahydro-quinolin-8-yl)-amino]-propyl}-amide;
N-{3-[(1H-benzimidazol-2-ylmethyl)-(5,6,7,8-tetrahydro-quinolin-8-yl)-amino]-propyl}-guanidine;
{3-[(1H-benzimidazol-2-ylmethyl)-(5,6,7,8-tetrahydroquinolin-8-yl)-amino]-propyl}-urea; and
{2-[(1H-benzimidazol-2-ylmethyl)-(5,6,7,8-tetrahydroquinolin-8-yl)-amino]-ethyl}-urea;

or a pharmaceutically acceptable salt thereof.

The following examples are offered to illustrate but not to limit the invention.

### Example 1

### Effects In Vivo of Multiple Dosing Strategies of AMD3465

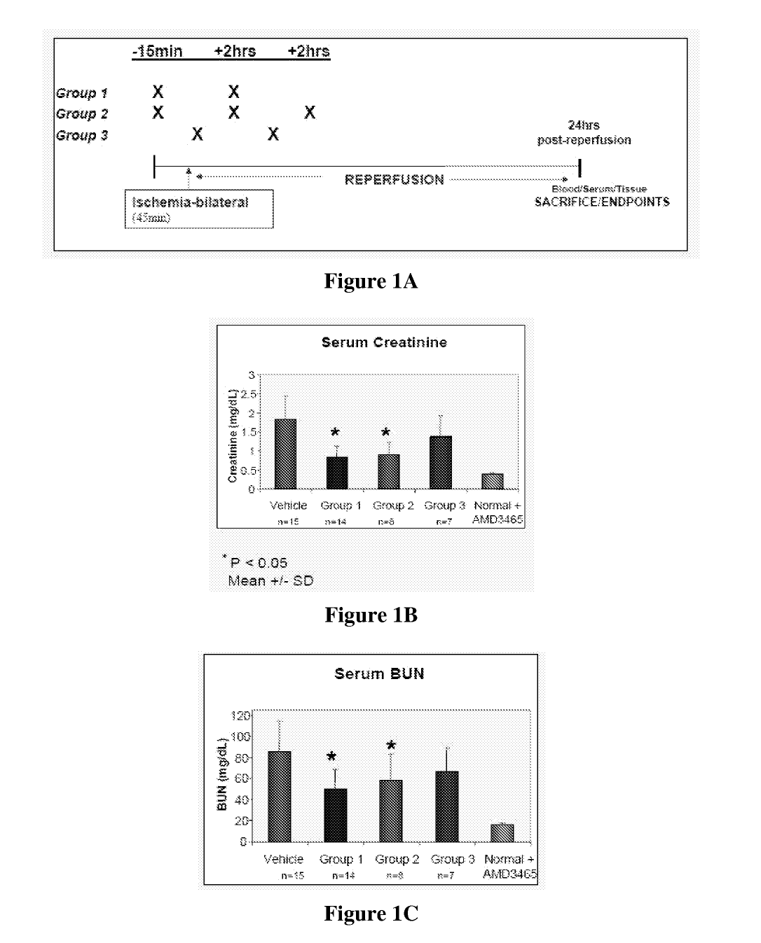

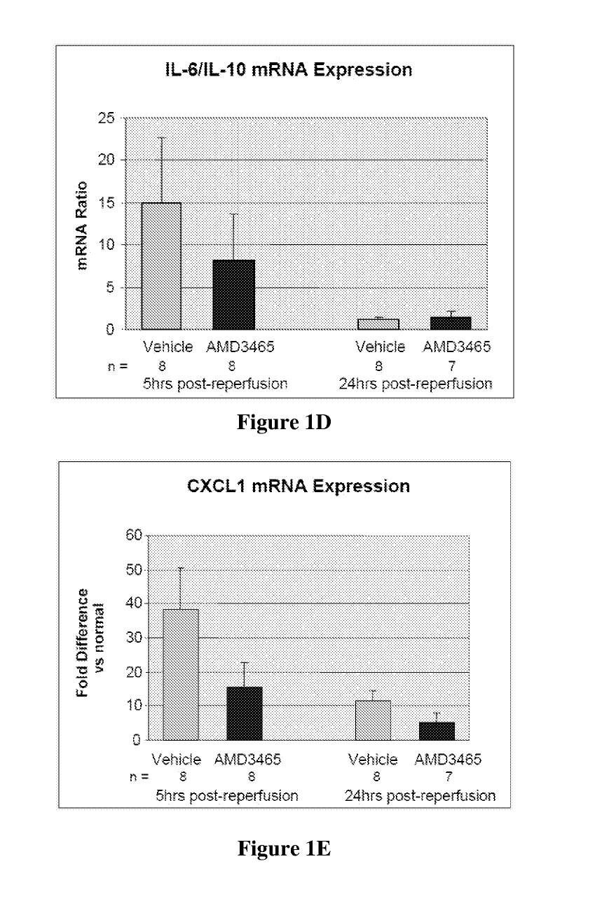

This example illustrates the effect of multiple dosing strategies of AMD3465 in an in vivo rat model of renal ischemia-reperfusion injury in which the renal artery and vein of both kidneys were clamped for 45 minutes followed by removal of the clamps and reperfusion with blood. Dosing AMD3465 at 10 mg/kg, 15 minutes prior to ischemia with another dose 2 hrs later, ameliorated the loss of renal function measured 24 hrs after reperfusion based on levels of serum creatine and serum blood urea nitrogen (BUN). Group 1 follows this dosing regimen as illustrated in FIG. 1A. FIGS. 1B and 1C show creatinine and BUN levels in mg/dL of Group 1 subjects compared to subjects following different dosing strategies shown in FIG. 1A using 10 mg/kg. Also shown in FIG. 1A—Group 2 was dosed at −15 min and +2 hrs and +4 hrs and Group 3 at time 0 and +2 hrs. Subjects administered with the vehicle that had undergone renal ischemia-reperfusion injury and normal subjects that did not undergo renal-ischemia reperfusion injury but received AMD3465 were compared to those with renal ischemia-reperfusion injury administered AMD3465. As shown in FIGS. 1B and 1C (* represents P<0.05.), group 1 and group 2 showed creatinine and BUN levels closest to normal 24 hrs after reperfusion.

FIGS. 1D and 1E show that the efficacy of AMD3465 in Group 1 rats (dosed as described in FIG. 1A) may be due to an anti-inflammatory effect. FIG. 1D shows that at 5 hrs post-reperfusion, the ratio of IL-6/IL-10 mRNA expression drops more drastically when AMD3465 is administered as compared to vehicle; expression was measured by RT-PCR. By 24 hrs post-reperfusion, the ratio has dropped in both cases.

FIG. 1E shows results for mRNA expression of CXCL1, the rat ortholog of human IL-8, in vehicle as compared to AMD3465 measured 5 hrs and 24 hrs post-reperfusion. Rats were dosed as described for Group 1 rats in FIG. 1A.

### Example 2

### Histological Effects

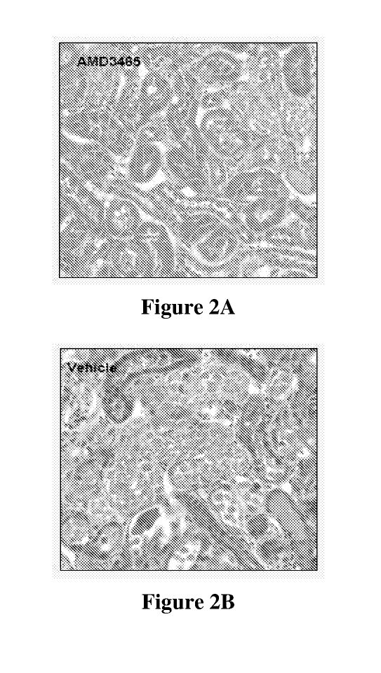

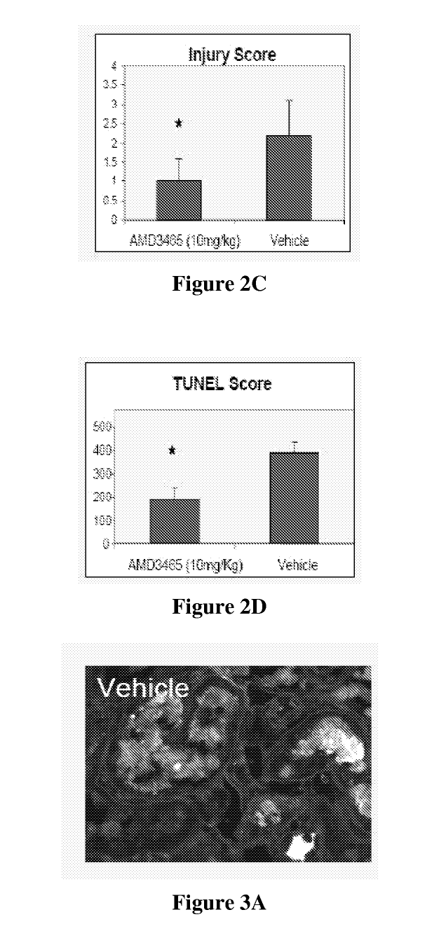

This example provides data showing that AMD3465 maintains structural integrity of the kidney. Rats were dosed as described for Group 1 rats in FIG. 1A. FIG. 2A represents a five micron histological section of kidney tissue from the 24 hr post-reperfusion timepoint stained with hematoxylin/eosin from a subject treated with AMD3465 that had undergone renal ischemia-reperfusion injury; FIG. 2B is an image of kidney tissue of a subject treated with the vehicle with renal ischemia-reperfusion injury. FIGS. 2C and 2D show graphs of the Injury and TUNEL score, respectively. Injury was evaluated by scoring hematoxylin and eosin stained kidney sections on a scale of 1 to 4 for proteinaceous cast formation, cell exfoliation, vascular congestion and loss of tubular architecture. TUNEL staining was carried out on kidney sections to identify apoptotic cells that were then quantified by Metamorph analysis. Comparisons between subjects treated with AMD3465 and subjects that were administered the vehicle revealed that AMD3465 maintained structural integrity by decreasing renal injury and cell death.

### Example 3

### Effect on Microvascular Permeability

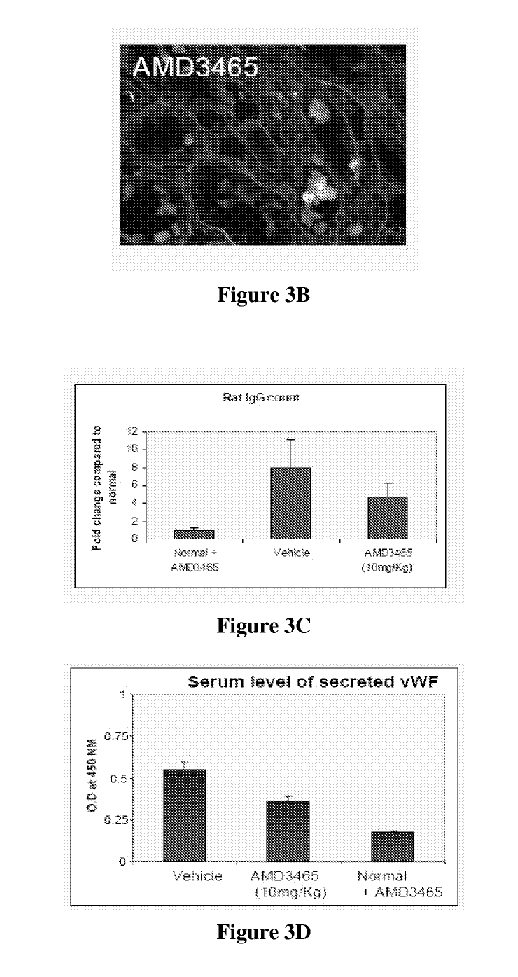

This example provides data that shows AMD3465 reduced microvascular permeability. Leakage of plasma proteins and secretion of von Willebrand Factor (vWF) are two indicators of the loss of microvascular integrity. FIGS. 3A and 3B are images of kidney tissue harvested 24 hrs after renal ischemia-reperfusion from subjects treated with the vehicle and AMD3465, respectively; tissue sections were immunostained with an antibody that recognizes rat immunoglobulin (IgG) and immunoreactivity quantified by Metamorph analysis illustrated in FIG. 3C. FIG. 3C shows the change of rat IgG count in subjects that had undergone renal ischemia-reperfusion injury and were administered either AMD3465 or vehicle, or normal subjects that received AMD3465. FIG. 3D shows the difference of vWF measured in the serum by ELISA from subjects that had undergone renal ischemia-reperfusion and were administered either AMD3465 or vehicle, or normal subjects that were administered AMD3465. Renal ischemia-reperfusion rats that received AMD3465 represent Group 1 rats in FIG. 1A. In rats that underwent renal ischemia-reperfusion and were treated with AMD3465, renal structure remained intact, with renal injury and cell death significantly reduced (FIG. 2C and FIG. 2D, respectively). Additionally, there was less damage to the peritubular capillary bed because frequency of serum proteins measured by rat IgG in the renal tubular lumen was less (FIGS. 3A, 3B, 3C) and circulating levels of the cleaved form of von Willebrand factor, an indicator of endothelial injury, was lower (FIG. 3D). The graphs indicate that administration of AMD3465 resulted in a reduction of IgG and secreted vWF, and suggests that AMD3465 helps reduce microvascular permeability in rats experiencing renal ischemia-reperfusion.

### Example 4

### AMD3465 is Well Tolerated

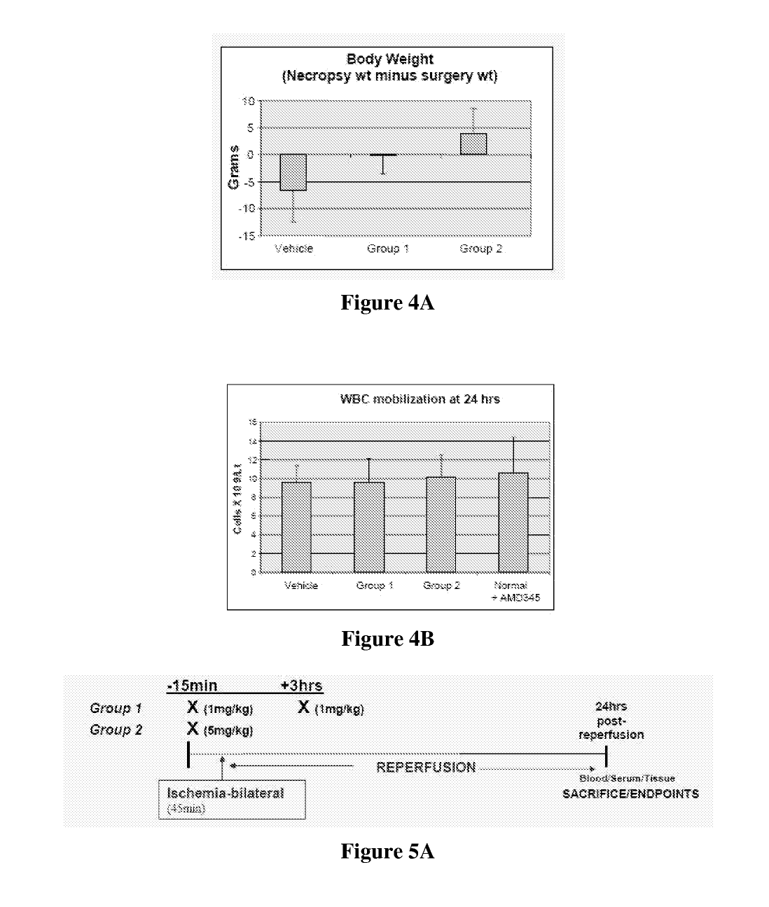

This example illustrates that AMD3465 is well tolerated in subjects treated with this compound that had undergone renal ischemia-reperfusion injury. FIG. 4A provides body weight data of subjects administered vehicle, and subjects in Group 1 and Group 2 (Groups described in FIG. 1A). Rats with renal ischemia-reperfusion injury and treated with AMD3465 lost less body weight than those subjects treated with vehicle. FIG. 4B gives white blood cell (WBC) counts at 24 hours of Group 1 and 2 subjects, subjects administered vehicle and normal subjects administered AMD3465. The data suggests that WBC counts were comparable 24 hours later between vehicle and AMD treated groups with or without renal ischemia-reperfusion injury.

### Example 5

### Effect of AMD3100 on Renal Function

This example shows that AMD3100 also ameliorates loss of renal function in AKI.

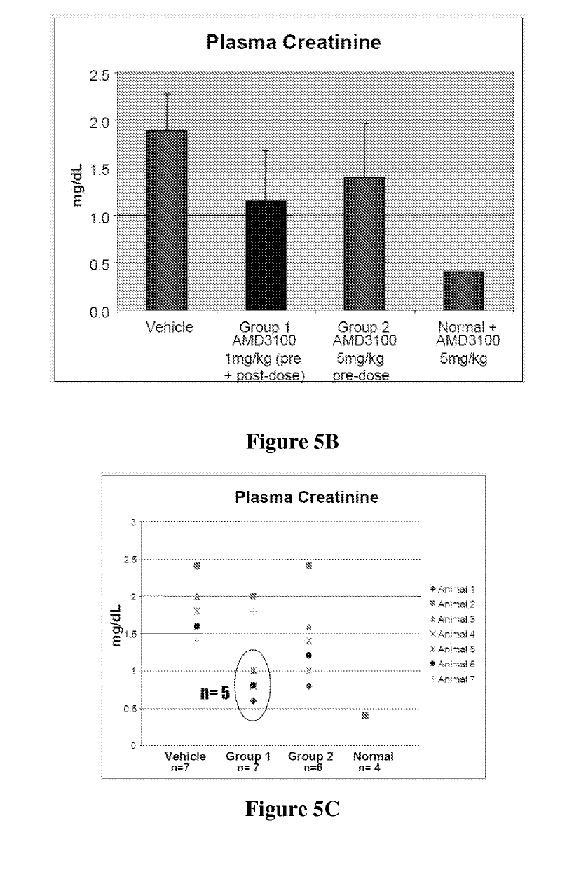

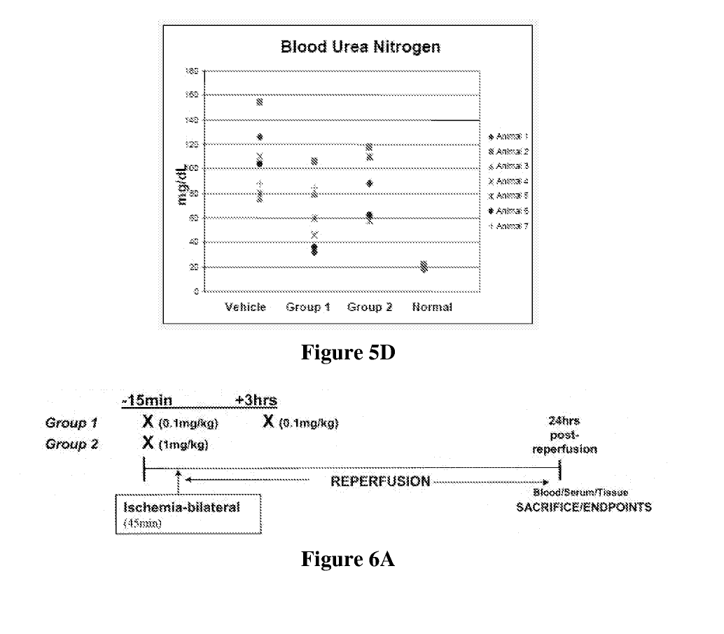

Based on data related to AMD3465, the in vivo efficacy of AMD3100 was tested. FIG. 5A diagrams the dosing regimen timeline for groups 1 and 2. FIGS. 5B, 5C and 5D show the creatine (serum or plasma) and blood urea nitrogen levels of the AMD treated groups, vehicle treated and normal subjects. Dosing was carried out with two 1 mg/kg administrations (Group 1) or 5 mg/kg in a single dose (Group 2). FIG. 5B demonstrates a decrease in plasma creatinine with two doses of 1 mg/kg (Group 1) and a single dose of 5 mg/kg (Group 2), albeit not significant (P>0.05). FIG. 5C and FIG. 5D illustrate individual data points of each rat for plasma creatinine and blood urea nitrogen, respectively. In FIG. 5C, five rats demonstrated lower plasma creatinine.

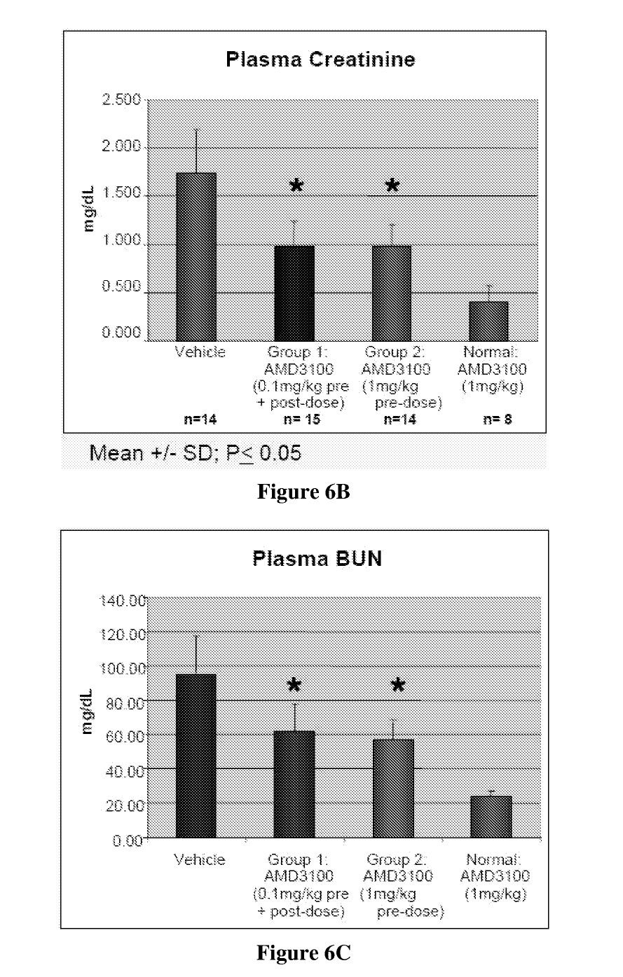

Lower doses were therapeutically effective as shown in FIGS. 6A-6C. FIG. 6A illustrates the dosing regimen. When administered 15 minutes prior to ischemia with another dose 3 hrs later at 0.1 mg/kg (Group 1) or only at 15 minutes prior to ischemia at 1 mg/kg (Group 2), AMD3100 was found to be therapeutically effective in ameliorating acute kidney injury. Both groups demonstrated amelioration of renal function loss (P≦0.05) This is supported by the reduction in plasma creatinine and BUN levels in subjects treated with AMD3100 shown in FIGS. 6B and 6C.

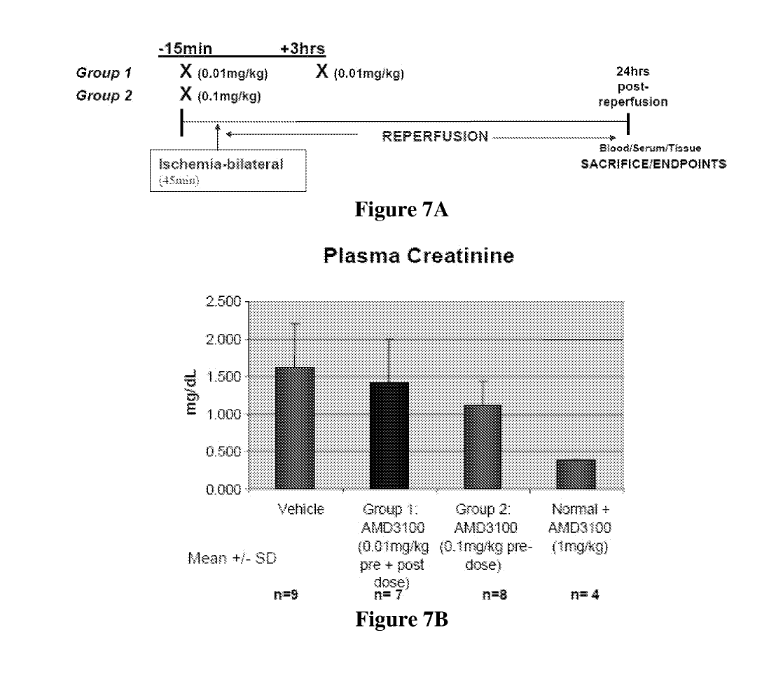

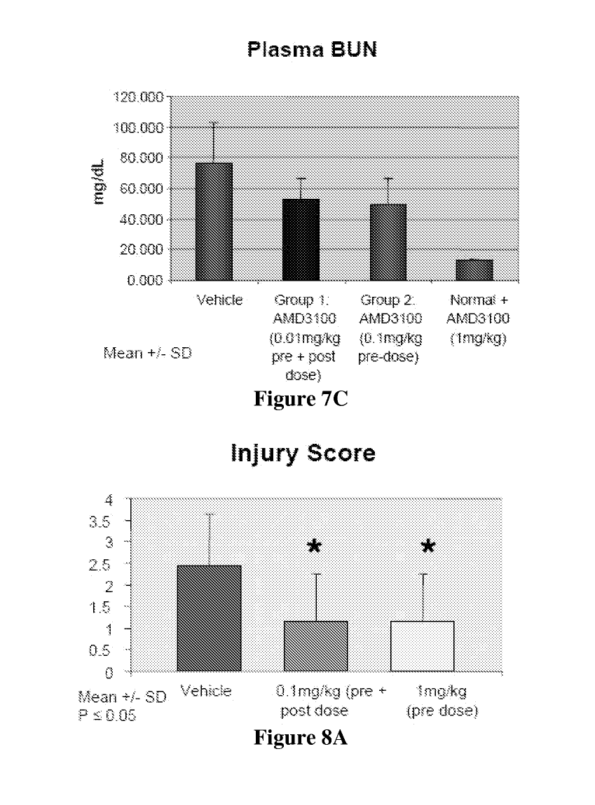

However, further lowering the dose resulted in some loss of effect as shown in FIGS. 7A-7C. FIG. 7A illustrates the dosing regimen. Group 1 was administered 0.01 mg/kg both 15 minutes before and 3 hours after ischemia; Group 2 was administered 0.1 mg/kg at 15 minutes before. FIG. 7B shows that while the effect on creatinine in Group 2 was comparable to that shown in FIG. 6B, Group 1 gave less positive results. The results with respect to plasma BUN in FIG. 7C are only slightly less effective than those in FIG. 6C.

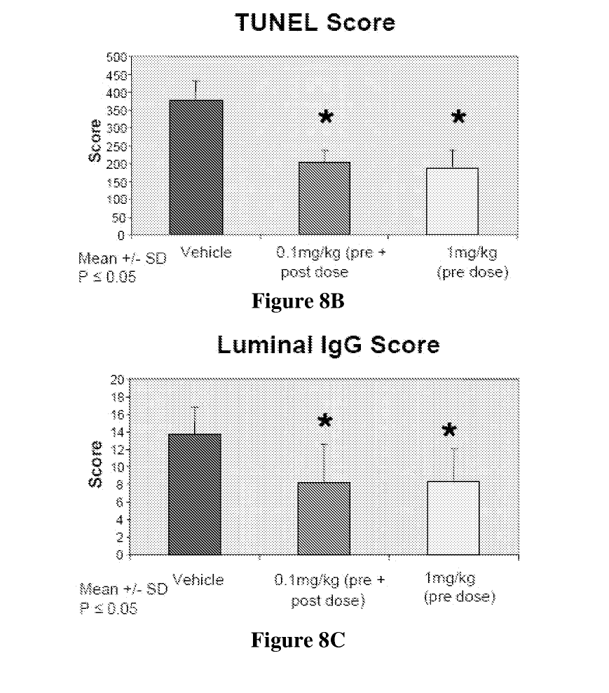

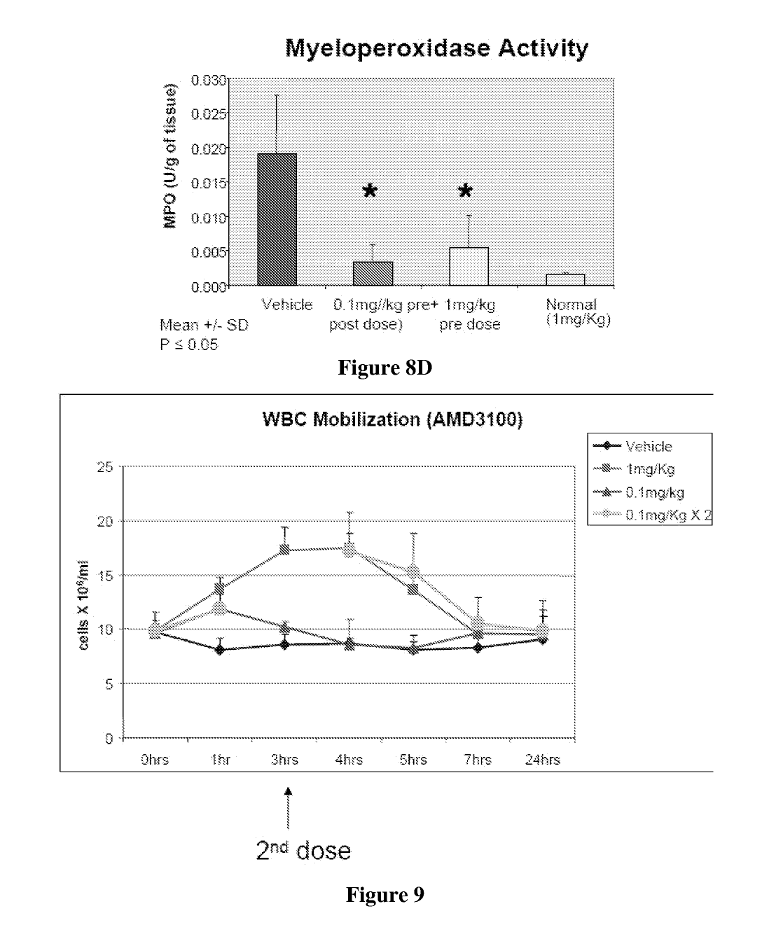

Using the regimen set forth in FIG. 6A wherein 0.1 mg/kg was administered 15 minutes before ischemia and 3 hours after or wherein only 1.0 mg/kg post-ischemia was administered, AMD3100 was able to lower epithelial injury and death and preserve vascular integrity. FIG. 8A shows the injury score lowered significantly as compared to vehicle and FIG. 8B shows a significant effect of TUNEL score. FIG. 8C shows a significant effect on luminal IgG score. Data for FIGS. 8A, 8B and 8C were generated as described for FIGS. 2C, 2D and 3C, respectively. FIG. 8D shows that for both regimens there was lowered myeloperoxidase activity, a measure of neutrophilic infiltration into the kidney tissue.

FIG. 9 shows the effect of AMD3100 on mobilization of white blood cells, used as an indicator of mobilization of stem cells. Comparable results were found in normal rats administered a single dose at time 0 of 1 mg/kg and 0.1 mg/kg administered twice, with the first dose given at time 0 and the second dose 3 hrs later.

Various documents including manuscripts, published patent applications, patents, etc. have been cited throughout this application and the contents of which are hereby incorporated herein by reference for all purposes.

1. A method for treating or preventing acute kidney injury (AKI) or a chronic form thereof in a subject in need thereof which comprises administering to said subject an effective amount of a compound of the formula
Z-linker-Z′ (1)
or pharmaceutically acceptable salt or prodrug form thereof
wherein Z is a cyclic polyamine containing 9-32 ring members of which 2-8 are nitrogen atoms, said nitrogen atoms separated from each other by at least 2 carbon atoms, and wherein said heterocycle may optionally contain additional heteroatoms besides nitrogen and/or may be fused to an additional ring system; 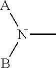
or Z is of the formula
wherein A comprises a monocyclic or bicyclic fused ring system containing at least one N and B is H or an organic moiety of 1-20 atoms,
Z′ may be embodied in a form as defined by Z above, or alternatively may be of the formula
—N(R)—(CR₂)ₙ—X
wherein each R is independently H or straight, branched or cyclic alkyl (1-6C), n is 1 or 2, and X is an aromatic ring, including heteroaromatic rings, or is a mercaptan or Z′ may be
—Ar(Y)ⱼ;
wherein Ar is an aromatic or heteroaromatic moiety, and each Y is independently a non-interfering substituent and j is 0-3; and
“linker” represents a bond, alkylene (1-6C) or may comprise aryl, fused aryl, oxygen atoms contained in an alkylene chain, or may contain keto groups or nitrogen or sulfur atoms.

2. The method of claim 1 wherein Z and Z′ are both cyclic polyamines.

3. The method of claim 1 wherein Z and Z′ are identical.

4. The method of claim 1 wherein Z and Z′ are both 1,4,8,11-tetraazocyclotetradecane.

5. The method of claim 4 wherein the linker is 1,4-phenylene-bis-methylene.

6. The method of claim 7 wherein the compound of formula (1) is 1,1′-[1,4-phenylene-bis-(methylene)-bis-1,4,8,11-tetraazacyclotetradecane, or a pharmaceutically acceptable salt or prodrug form thereof.

7. The method of claim 1 wherein
Z is of the formula 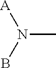
wherein A comprises a monocyclic or bicyclic fused ring system containing at least one N and B is H or an organic moiety of 1-20 atoms.

8. The method of claim 1 wherein Z′ is
—N(R)—(CR₂)ₙ—X
wherein each R, N and X are as defined in claim 1.

9. The method of claim 8 wherein the linker is 1,4-phenylene-bis-(methylene).

10. The method of claim 9 wherein Z′ is 2-aminomethyl-pyridine.

11. The method of claim 10 wherein the compound of formula (1) is N-[1,4,8,11-tetraazacyclotetradecanyl-1,4-phenylene-bis-(methylene)]-2-aminomethyl-pyridine or a pharmaceutically acceptable salt thereof.

12. The method of claim 1 wherein the compound of formula (1) is administered to said subject in the dosage range of about 0.1 μg/kg-5 mg/kg of body weight.

13. The method of claim 1 wherein the subject is human.

14. A method to reduce the risk of an allograft rejection in an allograft transplant recipient who receives an allograft from an allograft donor, which method comprises administering to said recipient an effective amount of a compound of the formula
Z-linker-Z′ (1)
or pharmaceutically acceptable salt or prodrug form thereof
wherein Z is a cyclic polyamine containing 9-32 ring members of which 2-8 are nitrogen atoms, said nitrogen atoms separated from each other by at least 2 carbon atoms, and wherein said heterocycle may optionally contain additional heteroatoms besides nitrogen and/or may be fused to an additional ring system;
or Z is of the formula 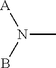
wherein A comprises a monocyclic or bicyclic fused ring system containing at least one N and B is H or an organic moiety of 1-20 atoms,
Z′ may be embodied in a form as defined by Z above, or alternatively may be of the formula
—N(R)—(CR₂)ₙ—X
wherein each R is independently H or straight, branched or cyclic alkyl (1-6C), n is 1 or 2, and X is an aromatic ring, including heteroaromatic rings, or is a mercaptan or Z′ may be
—Ar(Y)ⱼ;
wherein Ar is an aromatic or heteroaromatic moiety, and each Y is independently a non-interfering substituent and j is 0-3; and
“linker” represents a bond, alkylene (1-6C) or may comprise aryl, fused aryl, oxygen atoms contained in an alkylene chain, or may contain keto groups or nitrogen or sulfur atoms.

15. The method of claim 14 wherein the compound of formula (1) is 1,1′-[1,4-phenylene-bis-(methylene)-bis-1,4,8,11-tetraazacyclotetradecane, or a pharmaceutically acceptable salt or prodrug form thereof.

16. The method of claim 14 wherein the compound of formula (1) is N-[1,4,8,11-tetraazacyclotetradecanyl-1,4-phenylene-bis-(methylene)]-2-aminomethyl-pyridine or a pharmaceutically acceptable salt thereof.

17. A method to treat kidney disease in a subject in need of such treatment which method comprises administering to said subject an effective amount of 1,1′-[1,4-phenylene-bis-(methylene)-bis-1,4,8,11-tetraazacyclotetradecane, or a pharmaceutically acceptable salt or prodrug form thereof.

18. The method of claim 17, wherein the kidney disease is RPGN.
